(19) United States
(12) Patent Application Publication
(10) Pub. No.: US 2007/0166805 A1
(43) Pub. Date: Jul. 19, 2007

(54) Use of GR2 proteins to modify cellulosic materials and to enhance enzymatic and chemical modification of cellulose

(76) Inventors: Daniel J. Cosgrove, Port Matilda, PA (US); Takumi Takeda, State College, PA (US)

Correspondence Address: MCKEE, VOORHEES & SEASE, P.L.C.;ATTN: PENNSYLVANIA STATE UNIVERSITY, 801 GRAND AVENUE, SUITE 3200, DES MOINES, IA, 50309-2721, US

(21) Appl. No.: 11/655,354

(22) Filed: Jan. 19, 2007

Related U.S. Application Data
(60) Provisional application No. 60/760,185, filed on Jan. 19, 2006.

(51) Int. Cl. C12P 19/04; C12N 9/42; C12N 1/16; C12N 15/74

(52) U.S. Cl. 435/101; 435/209; 435/254.1; 435/484

(57) ABSTRACT

This invention concerns methods of use for a class of proteins heretofore known as “grass pollen group-⅔ allergens” to modify the properties of cellulose-based materials, changing their mechanical properties, improving their accessibility to agents that chemically modify cellulose structure (such as cellulytic enzymes and chemical reagents), and altering binding of dyes such as Direct Cotton dyes to cellulose.

### CROSS-REFERENCE TO RELATED APPLICATIONS

This application claims priority under 35 U.S.C. § 119 of a provisional application Ser. No. 60/760,185 filed Jan. 19, 2006, which application is hereby incorporated by reference in its entirety.

### GRANT REFERENCE

This work was supported by the Department of Energy pursuant to Grant No. 428-15 483X. Accordingly, the U.S. Government may have certain rights in the invention.

### BACKGROUND OF THE INVENTION

Cellulose is a polysaccharide that is synthesized in the form of a nano-scale microfibril. It is made mainly by plants and consists of many parallel strands of β-1,4-D-glucan bundled together to form a crystalline ribbon that is very strong and resists enzymatic attack (Hon & Shiraishi, 2000; Brown, Jr. et al., 1996). Cellulose makes up the fibrous component of plant cell walls and is the most abundant organic polymer on earth. It has widespread and diverse economic uses. As a fiber, it is used in textiles (cotton, linen, ramie, rayon are cellulosic fibers). Also paper is a matted sheet of cellulose fibers and lumber and other wood-based materials are principally cellulose in a less processed form. As a long organic polymer, it is used in chemically derivatized forms to make films and membranes (e.g. cellulose acetate, or “acetates” in the trade, nitrocellulose membranes, dialysis tubing, etc.) as well as thickeners and coatings (paints, lotions, nail polish, foods). As a polymer built of simple glucose (sugar) units, it is an important feedstock for ruminant animals and for fermentation-based processes for producing fuels such as ethanol. It also can be combusted directly for energy production. For biofuel (ethanol) production from cellulose, plant cell walls are collected and treated with hydrolytic enzymes such as fungal cellulase mixtures to break down the cellulose into simple sugars, which are subsequently fermented by yeasts to yield ethanol. The plant cell walls may be in the form of wood chips, corn residues (corn stover), sugarcane residues, waste paper and cardboard, and other materials derived from plants.

One of the major limitations and costs associated with ethanol production from cellulose is conversion of cellulose to simple fermentable sugars. Because of the crystalline structure of cellulose, its enzymatic conversion to sugars takes a considerable amount of time and requires large quantities of cellulase enzymes, which are expensive. Likewise for the production of chemically-modified cellulose derivatives, cellulose must be made accessible to reactive chemical agents, this usually requiring high temperature, pressures and harsh chemical conditions. Furthermore, the efficient digestion of straws, hay, and other plant materials by ruminants and other animals is limited by the accessibility of cellulose to the digestive enzymes in the animals' gut.

It is an object of the present invention to improve the efficiency of cellulase based reactions for use in production of bioethanol, cellulose derivitization, and/or enhanced digestion of plant feedstocks by ruminants and other animals.

It yet another object of the present invention to provide a biochemical agent which increases the accessibility of crystalline cellulose to attack by enzymes or chemical agents under standard conditions.

It is yet another object of the present invention to provide a composition for enhancing the cellulase activity of enzymes used in reactions with cellulose.

### SUMMARY OF THE INVENTION

The invention includes novel methods and compositions for the enhancement of the enzymatic degradation of cellulose. The composition comprises a GR2 protein and an enzyme having the property of degrading cellulose. In one embodiment, the enzyme is a hydrolytic enzyme. In a preferred embodiment, the enzyme is a cellulase. The cellulase may be derived from a variety of sources, including microorganisms such as Trichoderma.

The present invention further comprises a method for enhancing the accessibility of cellulose to chemical or biological modification. The method comprises incubating a sample containing cellulose with a GR2 protein. This method further comprises incubating the sample with a chemical or biological modifying agent. In one embodiment, the modifying agent comprises an enzyme having the property of degrading cellulose. In a preferred embodiment, the method comprises incubating the sample with a hydrolytic enzyme, such as cellulase.

The present invention also provides a method for enhancing enzymatic degradation of cellulose. The method of the invention comprises incubating a sample containing cellulose with a GR2 protein and an enzyme having the property of degrading cellulose. In one embodiment of the method the enzyme is a hydrolytic enzyme. In a preferred embodiment of the method, the enzyme is a cellulase. The cellulase may be derived from a variety of sources, including Trichoderma.spp, Aspergillus spp, Humicola spp and other fungi or bacteria.

With respect to the amounts of cellulose and GR2 present in the reaction mixture, in one embodiment the GR2 is present in an amount of at least about 0.0001 to about 0.005 times the amount of cellulose present in the reaction mixture. In a preferred embodiment, the GR2 is present in an amount of at least about 0.001 times the amount of cellulose present in the reaction mixture. With respect to the amounts of GR2 and cellulase utilized in the method of the present invention, in one embodiment the GR2 is present in an amount of at least about 0.01 to 0.5 times the amount of cellulase present. In a preferred embodiment, the GR2 is present in an amount of at least about 0.1 times the amount of cellulase present.

The novel method has commercial utility in various applications in that cellulose found in but not limited to sources such as textiles, paper and rope can be successfully treated using the method of the invention.

According to the invention, a biochemical agent has been found that increases the accessibility of crystalline cellulose to attack by enzymes or chemical agents under mild conditions of temperature, pressure and pH. The agent includes use of a class of proteins heretofore known as “grass pollen group-⅔ allergens” (Ansari et al., 1989b; Ansari et al., 1989a; Ansari et al., 1989c; Dolecek et al., 1993; Fedorov et al., 1997; De Marino et al., 1999) to modify the properties of cellulose-based materials, changing their mechanical properties, improving their accessibility to agents that chemically modify cellulose structure (such as cellulytic enzymes and chemical reagents), and altering binding of dyes such as Direct Cotton dyes to cellulose.

According to the invention, grass pollen group-⅔ allergens, hereafter referred to as GR2s, are small (˜10 kD), nonglycosylated proteins that are naturally and abundantly found in grass pollen, but are not evident in the pollen from other plant groups. They have 25-40% sequence identity with the carboxy-terminal domain of β-expansins, some of which have cell wall extension activity. The biological and biochemical functions of GR2s are mostly unknown, United States Published Application, 2004/0110190, the disclosure of which is herein incorporated by reference relates to the ability of GR2s to loosen cell walls in a β-expansin-like manner. The use of whole (two-domain) expansin proteins to enhance hydrolysis of cellulose by cellulases has been disclosed in U.S. Pat. No. 6,326,470 “Enhancement of accessibility of cellulose by expansins”. This patent discloses the use of whole expansins, which are very difficult to synthesize as active recombinant protein. The GR2 proteins, on the other hand, are readily expressed in recombinant systems. GR2 is much more stable than expansins, surviving extreme pHs, temperatures and organic solvents. In addition, the cellulase synergistic effects found for GR2 proteins are substantially greater than those found for expansins.

According to the invention, applicants herein show that GR2s lack cellulose hydrolytic activity by themselves, but when combined with any of a variety of cellulytic enzymes GR2s strongly enhance the hydrolysis of cellulose. The results indicate that GR2s to increase the accessibility of cellulose to enzymatic attack. GR2s also are shown herein to reduce the mechanical strength of paper and to affect binding of Direct Cotton dyes to cellulose. When combined with any cellulytic enzymes GR2s strongly enhance the hydrolysis of cellulose for any of a number of applications, including chemical derivitization of cellulose, bioethanol production, paper recycling, improvement of forage digestibility, and the like.

While not wishing to be bound by any theory it is postulated that the GR2 proteins modify the surface chains of cellulose, increasing the physical accessibility of cellulose to attack by cellulases. Any of a number of known cellulases may be used according to the invention including those listed herein and any other enzyme capable of hydrolyzing cellulose including chimeric proteins which include the functionally active domain of a cellulase enzyme.

### DESCRIPTION OF THE FIGURES

FIG. 1 depicts an alignment of the amino acid sequences of Zea m2 (top), Zea m3 (middle) and Lol p3 (bottom). Identical residues are shown in boxes.

FIG. 2 is a matrix showing the sequence identity between domain 2 of the GR2 protein (Zea m2, Zea m3, Lol p3). These all have similar cellulase synergistic activity although the homology is very low (15% down to 5%), as indicated in the matrix.

FIGS. 3A and B show the comparison of GR2 proteins sequences and sequence distances as percent identity and percent divergence. FIG. 3A is an alignment of 15 group-⅔ allergens by CLUSTAL W. The NCBI accession numbers for sequences are listed to the right. FIG. 3B is a table showing sequence distances as percent identity and percent divergence. Data calculated with the DNASTAR MEGALIGN program.

FIGS. 4A and 4B show the purification of native maize GR2. 4A. Elution from column, indicating where GR2 elutes. 4B. Coommassie-stained gel of the GR2 fraction fractionated by SDS-PAGE. Ten grams of frozen maize pollen was extracted in 40 mL of 50 mM sodium acetate, pH 4.5, for 1 hr at 4° C. The extract was centrifuged at 14,000 g at 4° C. and then loaded unto a CM-Sepharose column (15×300 mm) equilibrated in 20 mM sodium acetate, pH 4.5. The column was washed with the same buffer until absorbance at 280 nm returned to baseline. Protein was eluted at 1 mL/min. with a linear gradient of NaCl (0-500 mM in 6 h) Fractions were collected at 5 mL per tube. For SDS-PAGE, samples were de-salted before loading onto the gel. Large band at ˜10 kD=GR2, indicated with arrow.

FIG. 5 is a graph showing the time course for cellulytic release of soluble sugars from microcrystalline cellulose (Avicel) by Trichoderma cellulase +/− maize native GR2 protein. Striking enhancement of cellulase action by GR2 is observable. Left—Avicel (5 mg in 500 μl)) was incubated with Trichoderma cellulase (100 μg) +/− maize native GR2 (20 μg) at 30° C. for 1, 3 and 18 hours. The supernatant was used for measuring total soluble sugar by the anthrone method. Right: Avicel (5 mg) was incubated in sodium acetate buffer (0.5 ml, 50 mM, pH4.5) with EG1 (1 mg) and native GR2 protein (10 μg) at 30° C.

FIG. 6 is a graph showing that the maize GR2 synergism with different cellulase enzymes. Calibration: 1 microgram glucose=0.042 Absorbance 410 nm units. Methods: Avicel (10 mg/1.4 ml) was incubated with various cellulases (3.0 μg) and GR2 (20 μg) for 18 and 36 h at room temperature. GR2: open column fractionated protein from maize pollen.

FIGS. 7A and 7B are graphs showing that GR2 has little to no synergistic effect on hydrolysis of soluble glucans. A. Hydrolysis of CMC by EG1 cellulase +/−GR2. B. Hydrolysis of xyloglucan by Trichoderma cellulase +/− GR2. Calibration: 1 microgram glucose=0.042 Absorbance 410 nm units. Methods: 7A. Hydroxymethyl cellulose (2 mg/total 500 ml) in sodium acetate buffer (50 mM, pH 4.5) was incubated with EG1 (1 mg) and ZM3 (5 mg) for several hours at 30° C. A portion (40 ml) was removed and measured reducing power by PARBAR method. 7B. Reaction mixture (total 200 ml) containing 10 mg of Tamarind xyloglucan, 1 μg of Trichoderma cellulase, 50 mM sodium acetate (pH4.5) with or without group ⅔ protein (10 μg) was incubated at room temperature. A portion of the reaction mixture (50 ml) was used for measuring release of reducing sugars (measured as 410 nm absorbance, using the PAHBAH method).

FIG. 8 is a graph of GR2 synergism as a function of concentration. Avicel (5 mg) was incubated with Trichoderma reesei cellulase (50 μg), 50 mM sodium acetate (pH 4.5) and native GR2 protein (0-80 μg) at 30° C. for 3 and 18 hours. The supernatant was measured released sugars by anthrone/H₂SO₄ method.

FIGS. 9A and 9B show GR2 Synergism by recombinant ZM3. FIG. 9A is a graph of varying amounts of recombinant ZM3 that was added to Avicel (10 mg/1.4 ml) and incubated with EG1 cellulase (1.0 μg) for 18 h at room temperature. Release of reducing sugars (measured as absorbance at 410 nm) was assayed by the PAHBAH method. FIG. 9B is a gel showing the purity of recombinant ZM3 preparations. Soluble protein was extracted from E. coli expressing ZM3 protein, applied on CM Sepharose column and eluted with 10 mM sodium acetate plus 200 mM NaCl buffer. Five mg of Soluble protein (S), CM Sepharose column purified protein (P) and inclusion body (I) dissolved with 6M guanidine were subjected to SDS-PAGE (13% acrylamide gel), and stained with Coomassie blue.

FIG. 10 is a graph of GR2 synergism as a function of substrate concentration. Methods: Reaction mixture (500 μL) containing Avicel (0.5-10 mg), recombinant GR2 (5 μg) and EG1 (1 μg) was incubated at 30μ for 18 h. The supernatant was removed and measured released sugars by anthrone/H2SO4.

FIG. 11 is a graph showing the effect of temperature on GR2 synergism. Reaction mixture (500 μL) containing Avicel (5.0 mg), recombinant ZM3 (5 μg) and EG1 (1 μg) was incubated at 25. 30, 35 and 45 C for 18 h.

FIG. 12 is a graph depicting the effect of E1 cellulase concentration on GR2 synergism. Methods: A reaction mixture containing Avicel (5 mg), EG1 (1 mg), 50 mM sodium acetate (pH4.5) and recombinant ZM3 (5 mg) was incubated at 30° C. for 18 h. The supernatant was used for measuring released sugar. The figure was made based on the total sugar measurement by the anthrone assay.

FIG. 13 is a graph of the effect of pH on synergism of recombinant ZM3. Methods: Reaction mixture containing Avicel (5 mg), recombinant ZM3 (5 μg) and EG1 (1 μg) was incubated at 30 degree for 18 h. The supernatant was removed and measured released sugars by the anthrone method (total sugars released).

FIG. 14 is a graph showing the release of oligosacchrides from microfibrils by GR2 proteins. Methods: Avicel (10 mg/1.4 ml) was incubated with Trichoderma cellulase (140 μg) (open square) and GR2 (closed square) for 18 h at room temperature. Avicel treated with cellulase was boiled for 5 min and incubated with cellulase (140 μg) and/or GR2. GR2: open column fractionated protein from maize pollen.

FIG. 15 shows the measurement of the effects of GR2 on the mechanical strength of paper. Methods: 12 paper strips were cut (each treatment), 4 mm wide, 1.8 cm long; weight was equal. The strips were then soaked in 0.5 mL 20 mM Na acetate pH 5.5+/−140 ug Zm⅔ at 30° C. for 30 min; (20 uL). An Instron device was used to measure extension, 1.0 mm/min, 80 g force max, w/strip chart recorder. The figure shows 4 traces of force/extension curves. The first and third are of paper strips pretreated with GR2; the second and fourth are control strips.

### DETAILED DESCRIPTION OF THE INVENTION

Unless defined otherwise, all technical and scientific terms used herein have the same meaning as commonly understood by one of ordinary skill in the art to which this invention belongs. Unless mentioned otherwise, the techniques employed or contemplated herein are standard methodologies well known to one of ordinary skill in the art. The materials, methods and examples are illustrative only and not limiting. The following is presented by way of illustration and is not intended to limit the scope of the invention.

Units, prefixes, and symbols may be denoted in their ST accepted form. Unless otherwise indicated, nucleic acids are written left to right in 5′ to 3′ orientation; amino acid sequences are written left to right in amino to carboxy orientation, respectively. Numeric ranges are inclusive of the numbers defining the range. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes. The terms defined below are more fully defined by reference to the specification as a whole.

In describing the present invention, the following terms will be employed, and are intended to be defined as indicated below and herein.

By “altering physical characteristics of a plant cell wall” includes loosening or expanding cell walls, altering cell wall mechanical strength, altering the bonding relationship between the components of the cell wall and/or altering the growth of the plant cell wall.

By “encoding” or “encoded”, with respect to a specified nucleic acid, is meant comprising the information for translation into the specified protein. A nucleic acid encoding a protein may comprise non-translated sequences (e.g., introns) within translated regions of the nucleic acid, or may lack such intervening non-translated sequences (e.g., as in cDNA). The information by which a protein is encoded is specified by the use of codons. Typically, the amino acid sequence is encoded by the nucleic acid using the “universal” genetic code. However, variants of the universal code, such as is present in some plant, animal, and fungal mitochondria, the bacterium Mycoplasma capricolum (Proc. Natl. Acad. Sci. (USA), 82: 2306-2309 (1985)), or the ciliate Macronucleus, may be used when the nucleic acid is expressed using these organisms.

By “host cell” or “recombinantly engineered cell” is meant a cell which contains a vector and supports the replication and/or expression of the expression vector. Host cells may be prokaryotic cells such as E. coli, or eukaryotic cells such as yeast, Pichia. insect, plant, amphibian, or mammalian cells.

The present invention contemplates the use of expression vectors and host cells transformed to express the nucleic acid sequences encoding the GR2 proteins used in the methods and compositions of the invention. Nucleic acids coding for these GR2 proteins, or fragments thereof may be expressed in bacterial cells such as E. coli, fungi cells, plants or other systems for protein production. Suitable expression vectors, promoters, enhancers, and other expression control elements may be found in Sambrook et al. Molecular Cloning: A Laboratory Manual, second edition, Cold Spring Harbor Laboratory Press, Cold Spring Harbor, N.Y., 1989, which is incorporated herein by reference in its entirety.

Expression vectors are typically self-replicating DNA or RNA constructs containing the desired receptor gene or its fragments, usually operably linked to suitable genetic control elements that are recognized in a suitable host cell. These control elements are capable of effecting expression within a suitable host. The specific type of control elements necessary to effect expression will depend upon the eventual host cell used. Generally, the genetic control elements can include a prokaryotic promoter system or a eukaryotic promoter expression control system, and typically include a transcriptional promoter, an optional operator to control the onset of transcription, transcription enhancers to elevate the level of mRNA expression, a sequence that encodes a suitable ribosome binding site, and sequences that terminate transcription and translation. Expression vectors also usually contain an origin of replication that allows the vector to replicate independently of the host cell.

The vectors of this invention include those which contain DNA which encodes a protein, as described, or a fragment thereof encoding a biologically active equivalent polypeptide. The DNA can be under the control of a bacterial promoter and can encode a selection marker. Usually, expression vectors are designed for stable replication in their host cells or for amplification to greatly increase the total number of copies of the desirable gene per cell. It is not always necessary to require that an expression vector replicate in a host cell, e.g., it is possible to effect transient expression of the protein or its fragments in various hosts using vectors that do not contain a replication origin that is recognized by the host cell. It is also possible to use vectors that cause integration of the protein encoding portion or its fragments into the host DNA by recombination.

Expression vectors are specialized vectors which contain genetic control elements that effect expression of operably linked genes. Plasmids are the most commonly used form of vector but all other forms of vectors which serve an equivalent function and which are, or become, known in the art are suitable for use herein. See, e.g., Pouwels, et al. (1985 and Supplements) Cloning Vectors: A Laboratory Manual, Elsevier, N.Y., and Rodriguez, et al. (eds. 1988) Vectors: A Survey of Molecular Cloning Vectors and Their Uses, Buttersworth, Boston, which are incorporated herein by reference in their entirety.

The expression vectors listed herein are provided by way of example only of the well-known vectors available to those of ordinary skill in the art that would be useful to express the nucleic acid molecules. The person of ordinary skill in the art would be aware of other vectors suitable for maintenance propagation or expression of the nucleic acid molecules described herein. These are found for example in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989.

Prokaryotic host-vector systems include a wide variety of vectors for many different species. As used herein, E. coli and its vectors will be used generically to include equivalent vectors used in other prokaryotes. A representative vector for amplifying DNA is pBR322 or many of its derivatives. Vectors that can be used to express the polynucleotide or, its fragments include, but are not limited to, such vectors, as those containing the tac, ara, trp promoter, lac promoter, lacUV5 or T7 promoter. See Brosius, et al. (1988) “Expression Vectors Employing Lambda-, trp-, lac-, and Ipp-derived Promoters”, in Vectors: A Survey of Molecular Cloning Vectors and Their Uses, (eds. Rodriguez and Denhardt), Buttersworth, Boston, Chapter 10, pp. 205-236, which is incorporated herein by reference. Moreover, one skilled in the art knows that such microorganisms are available from depository authorities, e.g., the American Type Culture Collection (ATCC).

The use of promoter and cell type combinations for protein expression is generally known to those of skill in the art of molecular biology, for example, see Sambrook et al (1989). The promoters employed may be constitutive, or inducible, and can be used under the appropriate conditions to direct high level expression of the introduced DNA segment, such as is advantageous in the large-scale production of recombinant proteins or peptides.

Host cells can be transformed to express the nucleic acid sequences for use in the invention using conventional techniques such as calcium phosphate or calcium chloride co-precipitation, DEAE-dextran-mediated transfection, or electroporation. Suitable methods for transforming the host cells may be found in Sambrook et al. supra, which is incorporated by reference in its entirety and other laboratory textbooks.

The term “introduced” in the context of inserting a nucleic acid into a cell, means “transfection” or “transformation” or “transduction” and includes reference to the incorporation of a nucleic acid into a eukaryotic or prokaryotic cell where the nucleic acid may be incorporated into the genome of the cell (e.g., chromosome, plasmid, plastid or mitochondrial DNA), converted into an autonomous replicon, or transiently expressed (e.g., transfected mRNA).

The terms “polypeptide”, “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers. The term also includes variants, fragments or homologues thereof which retain biological activity of the polymer.

The term “residue” or “amino acid residue” or “amino acid” is used interchangeably herein to refer to an amino acid that is incorporated into a protein, polypeptide, or peptide (collectively “protein”). The amino acid may be a naturally occurring amino acid and, unless otherwise limited, may encompass known analogs of natural amino acids that can function in a similar manner as naturally occurring amino acids.

The term “expression cassette” refers to a polynucleotide sequence that comprises the coding sequence of interest and regulatory elements which affect expression of the protein of interest. Typically, expression cassettes include a promoter, the coding sequence of interest, a termination sequence, and a polyadenylation sequence. Optionally, expression cassettes can include enhancer elements and other regulatory elements.

The term “isolated nucleic acid” refers to a nucleic acid which is essentially free of other cellular components with which it is associated in the natural state. It is preferably in a homogeneous state although it can be in either a dry or aqueous solution. Purity and homogeneity are typically determined using analytical chemistry techniques such as gel electrophoresis or high performance liquid chromatography. The term “purified” denotes that a nucleic acid gives rise to essentially one band in an electrophoretic gel. Particularly, it means that the nucleic acid is at least 85% pure, more preferably at least 95% pure, and most preferably at least 99% pure.

The term “modifying or modification of cell walls” refers to changing the components, ratio of the components or structure of the components present in the cell wall, e.g., interference with the covalent interactions between cellulose microfibrils and matrix polysaccharides (McQueen-Mason, S. J. and Cosgrove, D. J. Plant Physiol. 107:87 (1995).

The term “operably linked” refers to functional linkage between a promoter and a second sequence, wherein the promoter sequence initiates transcription of RNA corresponding to the second sequence.

The sequence of the molecule can be defined herein in terms of homology to the exemplified sequence as well as in terms of the ability to hybridize with, or be amplified by, certain exemplified probes and primers. The polypeptides provided herein can also be identified based on their immunoreactivity with certain antibodies.

The GR2 polypeptides and polynucleotides for use in the subject invention can be identified and obtained by using oligonucleotide probes, for example, these probes are detectable nucleotide sequences. The probes (and the polynucleotides of the subject invention) may be DNA, RNA, or PNA (peptide nucleic acid). These sequences may be detectable by virtue of an appropriate label or may be made inherently fluorescent as described in International Application No. WO93/16094. As is well known in the art, if the probe molecule and nucleic acid sample hybridize by forming a strong bond between the two molecules, it can be reasonably assumed that the probe and sample have substantial homology. Preferably, hybridization is conducted under stringent conditions by techniques well-known in the art, as described, for example, in Keller, G. H., M. M. Manak (1987) DNA Probes, Stockton Press, New York, N.Y., pp. 169-170.

In a preferred method, one can use the BLAST search algorithm to identify polypeptides and oligonucleotides useful for the invention as well as other related sequences in a data set (such as a genome project or random sequencing of expressed cDNAs), followed by phylogenetic analysis with a group of known GR2 proteins (see Barry G. Hall, Phylogenetic Tree Made Easy, Sinauer, Sunderland Mass. 2001, 179 pp). This procedure robustly identifies GR2 proteins and differentiates them from expansins. GR2 proteins can also include active fragments of G.R2 proteins as well for example in the creation of chimeric proteins.

The term “polynucleotide,” “polynucleotide sequence” or “nucleic acid sequence” refers to deoxyribonucleotides or ribonucleotides and polymers thereof in either single- or double-stranded form. Unless specifically limited, the term encompasses nulcleic acids containing known analogues of natural nucleotides which have similar binding properties as the reference nucleic acid and are metabolized in a manner similar to naturally occurring nucleotides. Unless otherwise indicated, the nucleic acid sequence of this invention also implicitly encompasses conservatively modified variants thereof (e.g., degenerate codon substitutions) and complementary sequences and as well as the sequence explicitly indicated.

As used herein, the term “equivalent polypeptides” refers to polypeptides having the same or essentially the same biological activity as the claimed polypeptide.

As used herein, the terms “variants” or “variations” of genes refer to nucleotide sequences which encode the same polypeptides or which encode equivalent polypeptides.

Because of the redundancy of the genetic code, a variety of different DNA sequences can encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations” and represent one species of conservatively modified variation. Every nucleic acid sequence herein that encodes a polypeptide also describes every possible silent variation of the nucleic acid. One of ordinary skill will recognize that each codon in a nucleic acid (except AUG, which is ordinarily the only codon for methionine, one exception is Micrococcus rubens, for which GTG is the methionine codon (Ishizuka, et al., J. Gen'l Microbiol, 139:425-432 (1993)) can be modified to yield a functionally identical molecule. Accordingly, each silent variation of a nucleic acid, which encodes a polypeptide of the present invention, is implicit in each described polypeptide sequence and incorporated herein by reference.

It is well within the skill of a person trained in the art to create these alternative DNA sequences encoding the same, or essentially the same polypeptide. These variant DNA sequences are within the scope of the subject invention. As used herein, reference to “essentially the same” sequence refers to sequences which have amino acid substitutions, deletions, additions, or insertions which do not materially affect activity. Fragments retaining activity are also included in this definition.

For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations” and represent one species of conservatively modified variation. Other conservatively modified variants may be derived using related amino acids. Every nucleic acid sequence herein that encodes a polypeptide also describes every possible silent variation of the nucleic acid which encodes a polypeptide of the present invention, is implicit in each described polypeptide sequence and incorporated herein by reference.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” when the alteration results in the substitution of an amino acid with a chemically similar amino acid. Thus, any number of amino acid residues selected from the group of integers consisting of from 1 to 15 can be so altered. Thus, for example, 1, 2, 3, 4, 5, 7, or 10 alterations can be made. Conservatively modified variants typically provide similar biological activity as the unmodified polypeptide sequence from which they are derived. For example, substrate specificity, enzyme activity, or ligand/receptor binding is generally at least 30%, 40%, 50%, 60%, 70%, 80%, or 90%, preferably 60-90% of the native protein for it's native substrate.

The amino acid homology will be highest in critical regions of the polypeptides which account for biological activity or are involved in the determination of three-dimensional configuration which ultimately is responsible for the biological activity. In this regard, certain amino acid substitutions are acceptable and can be expected if these substitutions are in regions which are not critical to activity or are conservative amino acid substitutions which do not affect the three-dimensional configuration of the molecule. For example, amino acids may be placed in the following classes: non-polar, uncharged polar, basic, and acidic. Conservative substitutions whereby an amino acid of one class is replaced with another amino acid of the same type fall within the scope of the subject invention so long as the substitution does not materially alter the biological activity of the compound. Table A provides a listing of examples of amino acids belonging to each class.

TABLE A

| Class of Amino Acid | Examples of Amino Acids |
|---|---|
| Nonpolar | Ala, Val, Leu, Ile, Pro, Met, Phe, Trp |
| Uncharged Polar | Gly, Ser, Thr, Cys, Tyr, Asn, Gln |
| Acidic | Asp, Glw |
| Basic | Lys, Arg, His |

In some instances, non-conservative substitutions can also be made. The critical factor is that these substitutions must not significantly detract from the biological activity of the polypeptides.

Synthetic genes which are functionally equivalent to the polynucleotides of the subject invention can also be used to transform hosts. Methods for the production of synthetic genes can be found in, for example, U.S. Pat. No. 5,380,831. See also, Creighton (1984) Proteins W.H. Freeman and Company.

Equivalent polypeptides will have amino acid homology with exemplified polypeptides. The amino acid identity will typically be greater than 60%, preferably be greater than 70%, more preferably greater than 80%, more preferably greater than 90%, and can be greater than 95%.

As used herein, “reference sequence” is a defined sequence used as a basis for sequence comparison. A reference sequence may be a subset or the entirety of a specified sequence; for example, as a segment of a full-length cDNA or gene sequence, or the complete cDNA or gene sequence.

As used herein, “comparison window” includes reference to a contiguous and specified segment of a polynucleotide sequence, wherein the polynucleotide sequence may be compared to a reference sequence and wherein the portion of the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100, or longer. Those of skill in the art understand that to avoid a high similarity to a reference sequence due to inclusion of gaps in the polynucleotide sequence a gap penalty is typically introduced and is subtracted from the number of matches.

Methods of alignment of nucleotide and amino acid sequences for comparison are well known in the art. The local homology algorithm (Best Fit) of Smith and Waterman, Adv. Appl. Math may conduct optimal alignment of sequences for comparison. 2: 482 (1981); by the homology alignment algorithm (GAP) of Needleman and Wunsch, J. Mol. Biol. 48: 443 (1970); by the search for similarity method (Tfasta and Fasta) of Pearson and Lipman, Proc. Natl. Acad. Sci. 85: 2444 (1988); by computerized implementations of these algorithms, including, but not limited to: CLUSTAL in the PC/Gene program by Intelligenetics, Mountain View, Calif., GAP, BESTFIT, BLAST, FASTA, and TFASTA in the Wisconsin Genetics Software Package, Genetics Computer Group (GCG), 575 Science Dr., Madison, Wis., USA; the CLUSTAL program is well described by Higgins and Sharp, Gene 73: 237-244 (1988); Higgins and Sharp, CABIOS 5: 151-153 (1989); Corpet, et al., Nucleic Acids Research 16: 10881-90 (1988); Huang, et al., Computer Applications in the Biosciences 8: 155-65 (1992), and Pearson, et al., Methods in Molecular Biology 24: 307-331 (1994). The preferred program to use for optimal global alignment of multiple sequences is PileUp (Feng and Doolittle, Journal of Molecular Evolution, 25:351-360 (1987) which is similar to the method described by Higgins and Sharp, CABIOS, 5:151-153 (1989) and hereby incorporated by reference). The BLAST family of programs which can be used for database similarity searches includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences; and TBLASTX for nucleotide query sequences against nucleotide database sequences. See, Current Protocols in Molecular Biology, Chapter 19, Ausubel, et al., Eds., Greene Publishing and Wiley-Interscience, New York (1995).

GAP uses the algorithm of Needleman and Wunsch (J. Mol. Biol. 48: 443-453, 1970) to find the alignment of two complete sequences that maximizes the number of matches and minimizes the number of gaps. GAP considers all possible alignments and gap positions and creates the alignment with the largest number of matched bases and the fewest gaps. It allows for the provision of a gap creation penalty and a gap extension penalty in units of matched bases. GAP must make a profit of gap creation penalty number of matches for each gap it inserts. If a gap extension penalty greater than zero is chosen, GAP must, in addition, make a profit for each gap inserted of the length of the gap times the gap extension penalty. Default gap creation penalty values and gap extension penalty values in Version 10 of the Wisconsin Genetics Software Package are 8 and 2, respectively. The gap creation and gap extension penalties can be expressed as an integer selected from the group of integers consisting of from 0 to 100. Thus, for example, the gap creation and gap extension penalties can be 0, 1, 2, 3, 4, 5, 6, 7, 8, 9, 10, 15, 20, 30, 40, 50, or greater.

GAP presents one member of the family of best alignments. There may be many members of this family, but no other member has a better quality. GAP displays four figures of merit for alignments: Quality, Ratio, Identity, and Similarity. The Quality is the metric maximized in order to align the sequences. Ratio is the quality divided by the number of bases in the shorter segment. Percent Identity is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the Wisconsin Genetics Software Package is BLOSUM62 (see Henikoff & Henikoff (1989) Proc. Natl. Acad. Sci. USA 89:10915).

Unless otherwise stated, sequence identity/similarity values provided herein refer to the value obtained using the BLAST 2.0 suite of programs using default parameters. Altschul et al., Nucleic Acids Res. 25:3389-3402 (1997).

As those of ordinary skill in the art will understand, BLAST searches assume that proteins can be modeled as random sequences. However, many real proteins comprise regions of nonrandom sequences, which may be homopolymeric tracts, short-period repeats, or regions enriched in one or more amino acids. Such low-complexity regions may be aligned between unrelated proteins even though other regions of the protein are entirely dissimilar. A number of low-complexity filter programs can be employed to reduce such low-complexity alignments. For example, the SEG (Wooten and Federhen, Comput. Chem., 17:149-163 (1993)) and XNU (Claverie and States, Comput. Chem, 17:191-201 (1993)) low-complexity filters can be employed alone or in combination.

As used herein, “sequence identity” or “identity” in the context of two nucleic acid or polypeptide sequences includes reference to the residues in the two sequences, which are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g. charge or hydrophobicity) and therefore do not change the functional properties of the molecule. Where sequences differ in conservative substitutions, the percent sequence identity may be adjusted upwards to correct for the conservative nature of the substitution. Sequences, which differ by such conservative substitutions, are said to have “sequence similarity” or “similarity”. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Meyers and Miller, Computer Applic. Biol. Sci., 4: 11-17 (1988) e.g., as implemented in the program PC/GENE (Intelligenetics, Mountain View, Calif., USA).

As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “substantial identity” of polynucleotide sequences means that a polynucleotide comprises a sequence that has between 50-100% sequence identity, preferably at least 50% sequence identity, preferably at least 60% sequence identity, preferably at least 70%, more preferably at least 80%, more preferably at least 90% and most preferably at least 95%, compared to a reference sequence using one of the alignment programs described using standard parameters. One of skill will recognize that these values can be appropriately adjusted to determine corresponding identity of proteins encoded by two nucleotide sequences by taking into account codon degeneracy, amino acid similarity, reading frame positioning and the like. Substantial identity of amino acid sequences for these purposes normally means sequence identity of between 40-100%, preferably at least 55%, preferably at least 60%, more preferably at least 70%, 80%, 90%, and most preferably at least 95%.

Another indication that nucleotide sequences are substantially identical is if two molecules hybridize to each other under stringent conditions. The degeneracy of the genetic code allows for many amino acids substitutions that lead to variety in the nucleotide sequence that code for the same amino acid, hence it is possible that the DNA sequence could code for the same polypeptide but not hybridize to each other under stringent conditions. This may occur, e.g., when a copy of a nucleic acid is created using the maximum codon degeneracy permitted by the genetic code. One indication that two nucleic acid sequences are substantially identical is that the polypeptide, which the first nucleic acid encodes, is immunologically cross reactive with the polypeptide encoded by the second nucleic acid.

The terms “substantial identity” in the context of a peptide indicates that a peptide comprises a sequence with between 55-100% sequence identity to a reference sequence preferably at least 55% sequence identity, preferably 60% preferably 70%, more preferably 80%, most preferably at least 90% or 95% sequence identity to the reference sequence over a specified comparison window. Preferably, optimal alignment is conducted using the homology alignment algorithm of Needleman and Wunsch, J. Mol. Biol. 48: 443 (1970). An indication that two peptide sequences are substantially identical is that one peptide is immunologically reactive with antibodies raised against the second peptide. Thus, a peptide is substantially identical to a second peptide, for example, where the two peptides differ only by a conservative substitution. In addition, a peptide can be substantially identical to a second peptide when they differ by a non-conservative change if the epitope that the antibody recognizes is substantially identical. Peptides, which are “substantially similar” share sequences as, noted above except that residue positions, which are not identical, may differ by conservative amino acid changes.

The term “transgenic plant” refers to a plant into which exogenous polynucleotides have been introduced by any means other than sexual cross or selfing. Examples of means by which this can be accomplished are described below, and include Agrobacterium-mediated transformation, biolistic methods, electroporation, in planta techniques, and the like. Such a plant containing the exogenous polynucleotides is referred to here as an R₁ generation transgenic plant. Transgenic plants may also arise from sexual cross or by selfing of transgenic plants into which exogenous polynucleotides have been introduced.

The term “selectively hybridizes” includes reference to hybridization, under stringent hybridization conditions, of a nucleic acid sequence to a specified nucleic acid target sequence to a detectably greater degree (e.g., at least 2-fold over background) than its hybridization to non-target nucleic acid sequences and to the substantial exclusion of non-target nucleic acids. Selectively hybridizing sequences typically have about at least 40% sequence identity, preferably 60-95% sequence identity, and most preferably 100% sequence identity (i.e., complementary) with each other.

The terms “stringent conditions” or “stringent hybridization conditions” include reference to conditions under which a probe will hybridize to its target sequence, to a detectably greater degree than other sequences (e.g., at least 2-fold over background). Stringent conditions are sequence-dependent and will be different in different circumstances. By controlling the stringency of the hybridization and/or washing conditions, target sequences can be identified which can be up to 100% complementary to the probe (homologous probing). Alternatively, stringency conditions can be adjusted to allow some mismatching in sequences so that lower degrees of similarity are detected (heterologous probing). Optimally, the probe is approximately 500 nucleotides in length, but can vary greatly in length from less than 500 nucleotides to equal to the entire length of the target sequence.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide or Denhardt's. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C. Specificity is typically the function of post-hybridization washes, the critical factors being the ionic strength and temperature of the final wash solution. For DNA-DNA hybrids, the Tₘ can be approximated from the equation of Meinkoth and Wahl, Anal. Biochem., 138:267-284 (1984): Tₘ=81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization and/or wash conditions can be adjusted to hybridize to sequences of the desired identity. For example, if sequences with ≧90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3, or 4° C. lower than the thermal melting point (Tₘ); moderately stringent conditions can utilize a hybridization and/or wash at 6, 7, 8, 9, or 10° C. lower than the thermal melting point (Tₘ); low stringency conditions can utilize a hybridization and/or wash at 11, 12, 13, 14, 15, or 20° C. lower than the thermal melting point (Tₘ). Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution) it is preferred to increase the SSC concentration so that a higher temperature can be used. An extensive guide to the hybridization of nucleic acids is found in Tijssen, Laboratory Techniques in Biochemistry and Molecular Biology—Hybridization with Nucleic Acid Probes, Part I, Chapter 2 “Overview of principles of hybridization and the strategy of nucleic acid probe assays”, Elsevier, N.Y. (1993); and Current Protocols in Molecular Biology, Chapter 2, Ausubel, et al., Eds., Greene Publishing and Wiley-Interscience, New York (1995). Unless otherwise stated, in the present application high stringency is defined as hybridization in 4×SSC, 5×Denhardt's (5 g Ficoll, 5 g polyvinypyrrolidone, 5 g bovine serum albumin in 500 ml of water), 0.1 mg/ml boiled salmon sperm DNA, and 25 mM Na phosphate at 65° C., and a wash in 0.1×SSC, 0.1% SDS at 65° C., two to three times for at least 15 minutes.

Purification of GR2 Allergens

Purification of β-expansins and GR2 allergens from ryegrass pollen involved three successive chromatographic steps as depicted the references described supra. During the purification steps, fractions from each step were examined for expansin-like proteins by wall extension assay in combination with SDS-PAGE. The starting material was crude extract obtained from commercial ryegrass pollen with 50 mM sodium acetate, pH 4.5. On the conventional SP-Sepharose cation exchange chromatographic column the proteins with expansin-like activity were well separated from unbound impurities, yielding a sharp peak which predominantly contained expansin activity-like proteins. The fractions under this peak were pooled, desalted/concentrated through a 5 kD cutoff filtration membrane, and then chromatographed on a CM-silica based HPLC column.

The term “cellulase” encompasses any enzyme which hydrolyzes cellulose into smaller units and includes cellulases derived from microbes and other widely available sources. See Gilbert H J, Hazlewood G P, 1993 J Gen Microbiol 139:187-194; Olimiya K et al. 1997 Biotechnol Genet Eng Rev. 14:365-414.

“Modification or degradation of cellulose” is used herein to mean the chemical, biological or enzymatic induced alteration of the native structure of the cellulose. Cellulose is a polysaccharide of glucose units (C₆H₁₀O₅). Such changes and alterations, are known to those in the art and include those involved in enzymatic degradation and/or hydrolysis of cellulose, as well as chemical modifications involved in a variety of commercial cellulose based products such as textiles, cellulose acetate used in film, nitrocellulose, thickening agents such as those used in paint, papers/pulps, membranes, production of alcohols by fermentation of biomass, and other commercial applications. See Lapasin R. and S Priel. 1995 The Rheology of Industrial Polysaccharides. Theory and Application. London Blackie Academic and Professional; and M. E. Himmel et al., 1999 Curr Opin Biotechnol 10(4):358-364.

According the invention, GR2 proteins make cellulose more accessible to the degradative action of cellulases, thereby synergizing or enhancing the activity of cellulases. Cellulase activity can be measure in a number of ways including measuring the total amount of reducing sugars produced in the enzymatic reaction the cell. For other methods see Wood T M and K M Bhat 1988 Meth Enzymol 160:87-112. Expansins increase both the rate and total amount of degradation of cellulose. It is contemplated that this newly discovered effect of GR2 proteins can enhance the accessibility of cellulose for chemical, biochemical, or biophysical modification.

Other methods of treatment or use of specific GR2 proteins, enzymes possessing expansin-like activity, or genetically modified organisms possessing GR2 encoding gene(s) or gene(s) that have GR2-like activity may modify cellulose by a number of various actions, including but not limited to chemical, biochemical, or biophysical activity. Such variations are anticipated to have numerous commercial utilities, which may include but not be limited to the following, the modification or alteration of cellulose in the production of commercial products or intermediates utilized in production of paper/pulp, textile, fodder, biomass alcohol, films, coatings, thickeners, agricultural and/or food applications. See M. E. Himmel et al., 1999 Curr Opin Biotechnol 10(4):358-364.

In another embodiment the invention involves the creation of chimeric proteins which can include the active domains of GR2 proteins and the active domain of cellulase. Methods for creating these types of chimeras are known in the art as well as the active regions of GR2 and cellulase enzymes which would be desirable to have in a chimeric protein.

For commercial purposes, purified or crude GR2 proteins could be added to cellulose preparations in processes and procedures where cellulose accessibility is a limiting factor or where alterations in cellulose surface glucans could give desirable properties or traits. Enhanced production of biofuels, ethanol or other low molecular weight substances from cellulose is a likely potential target for this application of GR2 proteins, whether by cellulase or some other enzyme system. Likewise, processes that chemically derivatize cellulose may be made more efficient by the application of GR2 proteins to cellulose. GR2 could be added in purified or unpurified form to the cellulose, or it could be engineered by standard methods to be expressed in plants or other organisms, which then might be added whole to the cellulose preparations, e.g. for biofuels production.

Alteration of the specific GR2, cellulase, and source and type of cellulose are within the scope of this invention. Such variation may have significant, commercial utility. These results may vary because of, but not limited to, the following factors: substrate specifications, solubilities, pH optima, and other biochemical and/or biophysical properties, but GR2 proteins in concert with cellulase will nonetheless enhance or synergistically impact cellulose degradation.

What is presented here is a method using GR2(s), enzymes possessing GR2-like activity or genetically modified organisms possessing GR2 gene(s) or genes that have GR2-like activity in the presence of a cellulase to modify cellulose by a number of various actions, including but not limited to chemical, biochemical, or biophysical activity.

Definition of GR2 Proteins

Although GR2 allergens from grass pollen have been studied for many years by immunologists concerned with how they elicit hay fever and related allergic responses in humans, the native activity and biological roles of these proteins have not been examined. GR2 grass pollen allergens are distinguished by pI and immuno-cross reactivity, but accumulating sequence information indicates that they belong to the same protein family, which is referred to herein as GR2 allergens. Genes for GR2 allergens encode a protein with a signal peptide and a mature protein with statistically significant sequence similarity (up to 42% identity) with domain 2 of expansins, with the greatest similarly to group-1 allergen sub-class of β-expansins. Expansins are plant cell wall-loosening proteins involved in cell growth, leaf primordium initiation, fruit softening, pollen-tube penetration of the stigma, cell wall disassembly, and other developmental processes where wall loosening may be important.

Two families, α- and β- of expansins are currently recognized, and GR2 allergens are closest in sequence to the subset β-expansins known to immunologists as the grass pollen group 1 allergens. In the α- and β-expansin families, the proteins comprise two domains, an N-terminal domain, homologous with the catalytic domain of glycosyl hydrolase family 45 (GH45) enzymes, and a C-terminal domain with an immunoglobulin-like beta-sandwich fold.

The characteristic action of expansin on cell wall extensibility and stress relaxation is due to the action of domain 2, at least in part. However, except for the single case of the grass GR2 pollen allergens, the GH45-like domain 1 of expansin has apparently been preserved in this group of genes throughout plant evolution (˜500 million years). This in turn indicates that the GH45-like domain has an important role in expansin function.

For the purposes of this invention, the tern “GR2 protein” shall include but not be limited to the descriptions herein. One description includes all single-domain proteins that are homologous to domain 2 of the expansin family. Sampedro and Cosgrove (Sampedro & Cosgrove, 2005) define domain-2 of expansin superfamily, based on BLAST analysis and phylogenetic methods. The GR2 class also corresponds to family 01357 in the Pfam database of protein families (www.pfam.wustl.edu/index.html), which are defined based on hidden Markoff methods (Durbin et al., 1998). As of January, 2006, 476 distinct protein sequences were identified in the Pfam 01357 group. The GR2 class specifically includes
a) native forms of group-2 and group-3 grass pollen allergens (De Marino et al., 1999; Dolecek et al., 1993; Ansari et al., 1989c; Ansari et al., 1989b);
b) recombinant forms of (a);
c) proteins and protein fragments derived from expansin domain-2, including natural and recombinant sources of expansins, where the term “expansin” refers to the expansin superfamily as defined by Sampedro and Cosgrove (Sampedro & Cosgrove, 2005);
d) recombinant protein engineered to contain domain-2 from the expansin superfamily, including the EXPA, EXPB, EXLA, EXLB and EXLX groups of proteins, as defined by Kende et al. (Kende et al., 2004).
e) Proteins identified in Pfam 01357 and their sequence variants.

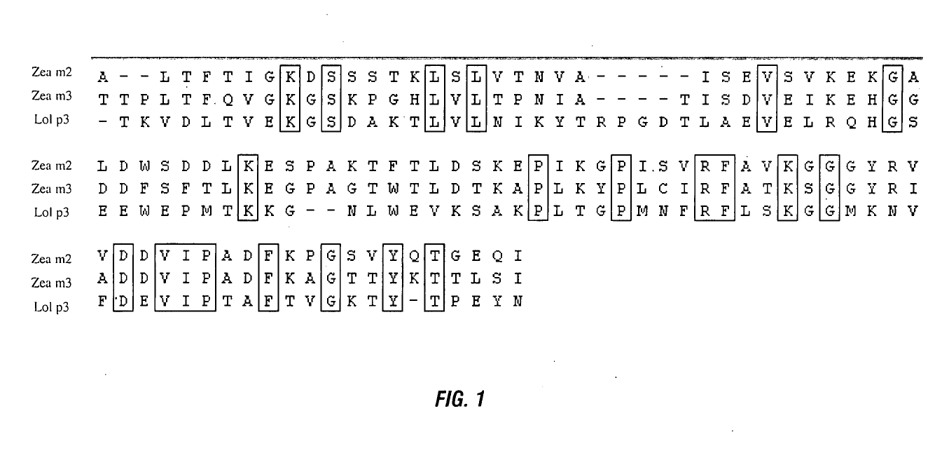

Examples of (a), the group-2 and group-3 grass pollen allergens, include Lol p2 (NCBI Protein Accession #P14947), Lol p3 (P14948), Phl p2 (P43214), Zea m2 (sequence #1, FIG. 1), and Zea m3 (sequence #2, FIG. 1). This list is not intended to be complete, but is representative of the natural proteins within this group. Other examples of GR2 class are identified in the listing of sequences for Pfam 01357 (see www.pfam.wust1.edu/cgi-bin/getdesc?name=Pollen_allerg_{—}1).

The present invention is broadly applicable to alteration of various physical properties of polysaccharides. While plant polysaccharides represent a preferred embodiment of the invention, GR2 proteins of the invention are useful catalytic proteins for the treatment polysaccharides from a variety of sources (i.e., synthetic, bacterial or other microbial system, etc.). The GR2 protein may also include a fragments or variations thereof which retains the cellulase enhancing activity of the protein.

GR2 proteins may be used for de-inking paper, which is a significant limitation in current paper recycling operations. In this application, GR2 proteins may help loosen the bonding between surface polymers, which are stuck to the ink, and the remainder of the cellulose fibers. Also, in the paper industry Gr2 proteins may prove useful for large scale paper dissolution, or perhaps for alteration of the mechanical properties of dry paper. Treatment of dry paper could produce paper with novel properties.

GR2 proteins may be used in combination with other chemicals or enzymes to improve various processes. For example, a major limitation in ethanol production from biomass is the degradation of cellulose. GR2s in concert with cellulase act synergistically in the breakdown of cellulose. Also, delignification is a major problem in industrial uses of many plant fibers. To solve this concern, GR2 proteins may be used with lignases (peroxidases) for synergistically de-lignifying plant fibers. GR2 proteins may also be useful in various bioremediation applications, either above or in combination with other biological or chemical materials.

A further use of GR2 proteins could be for altering the mechanical strength of gels or otherwise affecting the gelling or other properties of gels in combination with other enzymes (i.e., gelatin, gellum-gum/phytagel, agar, agarose, etc.). Such material are useful agents in foods, cosmetics, and other similar materials. Since hydrogen bonding is important for such gels and in view of the belief that Gr2 proteins, like expansins alter hydrogen bonding of wall glycans, GR2 may alter the gelling properties of various gels.

An additional use of GR2 proteins may involve alteration of aggregation of hemicellulose, a solubilized cellulose. In the event that GR2 proteins appropriately affect such aggregation, they may prove useful for industrial processes involving these materials, including cellulose processing and film making.

Because the GR2 proteins lack cysteines and are active when expressed in bacterial cells, this form of expansin-like protein is a much better candidate for large-scale production and commercialization than α- and β-expansins, which are difficult to express in recombinant protein expression systems. Using standard methods of genetic engineering, which are well known to those of ordinary skill, to express these proteins in bacteria, fungi, and plants, or other systems for protein production, it is possible to produce large quantities of these proteins for various applications anticipated for expansins, for example, altering the properties of cellulose-based materials such as paper, wood, textiles; wood fiber degradation and biofuel production; and synergistic enhancement of cellulase activity.

Furthermore, because GR2 proteins lack cysteines, they are likely to be more stable in commercial applications, i.e., not sensitive to thiol oxidation and inactivation by traces of metals such as mercury, copper, and other oxidative catalysts.

According to the invention, applicants have discovered cellulase synergistic activity in the GR2 proteins Zea m2, Zea m3 and Lol p3. These proteins are very divergent, with only 22% identity between Zea m2 and Lol p3. See illustration in FIG. 1 for alignment of Zea m2 (top), Zea m3 (middle) and Lol p3 (bottom). Identical residues are shown in boxes.

Although sequence identity among GR2 proteins is low, it is expected that all GR2 proteins will have the same protein structure, as represented by the structure determined for Phl p2 (De Marino et al., 1999). From the results herein, and from the well-based assumption that proteins within a Pfam family will have the same protein structure (Durbin et al., 1998), one would expect that all of the GR2 proteins should have the activities identified below for Zea m2, Zea m3 and Lol p3.

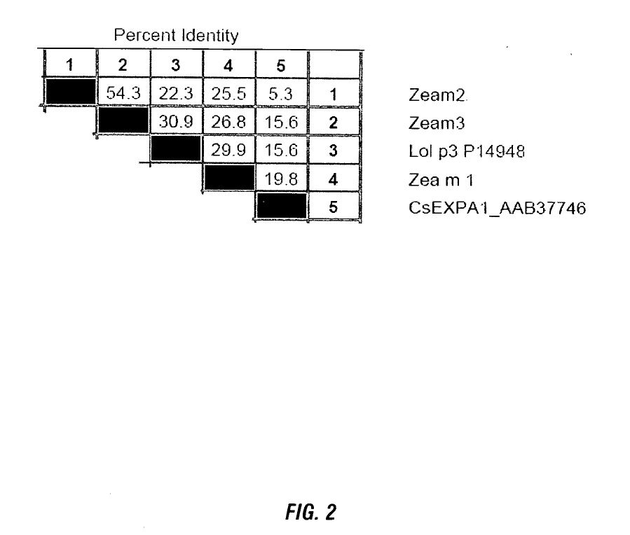

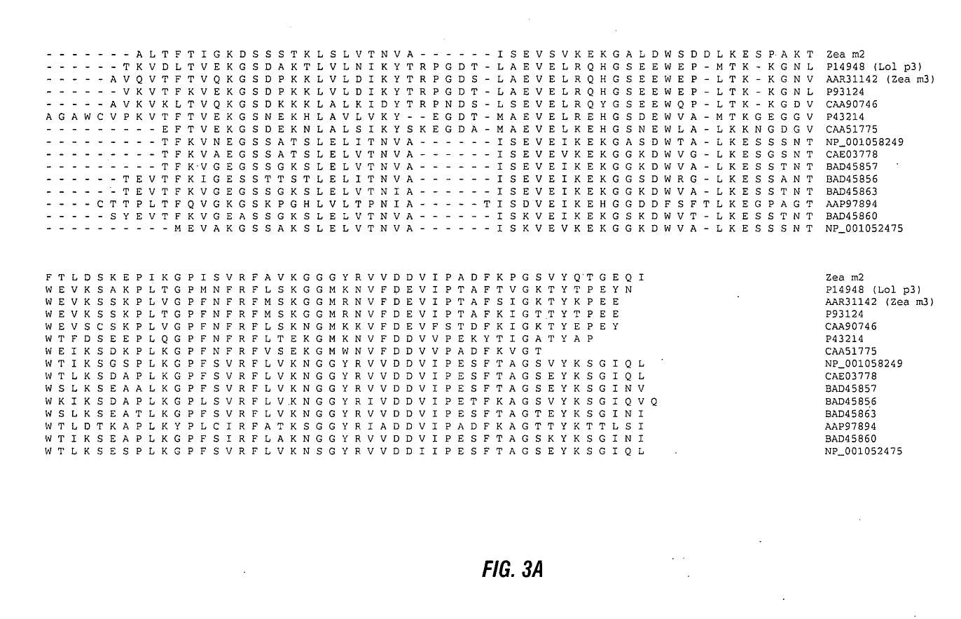

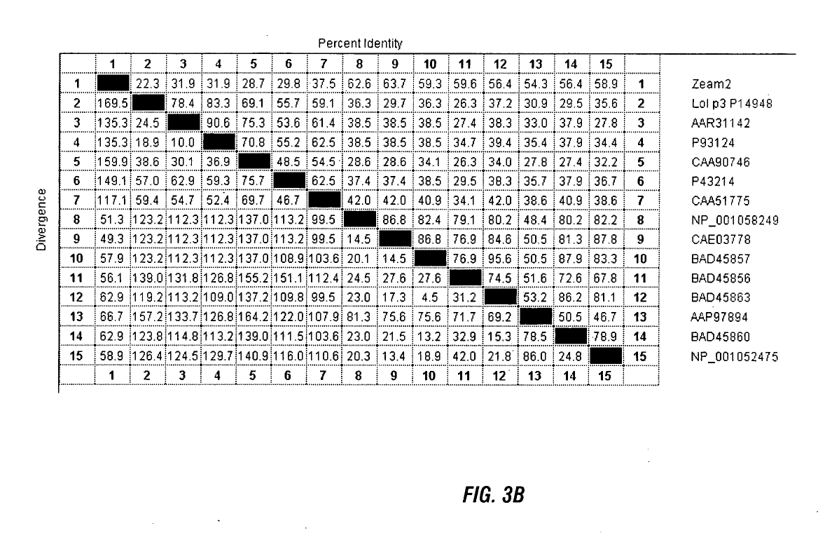

We also note that cellulase synergism was previously reported in U.S. Pat. No. 6,326,470 “Enhancement of accessibility of cellulose by expansins”, which demonstrated cellulase synergism for expansins. It can be thus concluded that domain-2 of these maize and cucumber expansins was responsible for the cellulase synergism reported in U.S. Pat. No. 6,326,470. The sequence identity between these domains and the GR2 protein (Zea m2, Zea m3, Lol p3) with similar cellulase synergistic activity is indeed very low (15% down to 5%), as indicated in the matrix shown in FIG. 2. This line of evidence adds further confidence that most, if not all, GR2 proteins are likely to exhibit cellulase synergism. Despite the low sequence homology, the functional cellulase synergy activity of the proteins remained.

### EXAMPLES

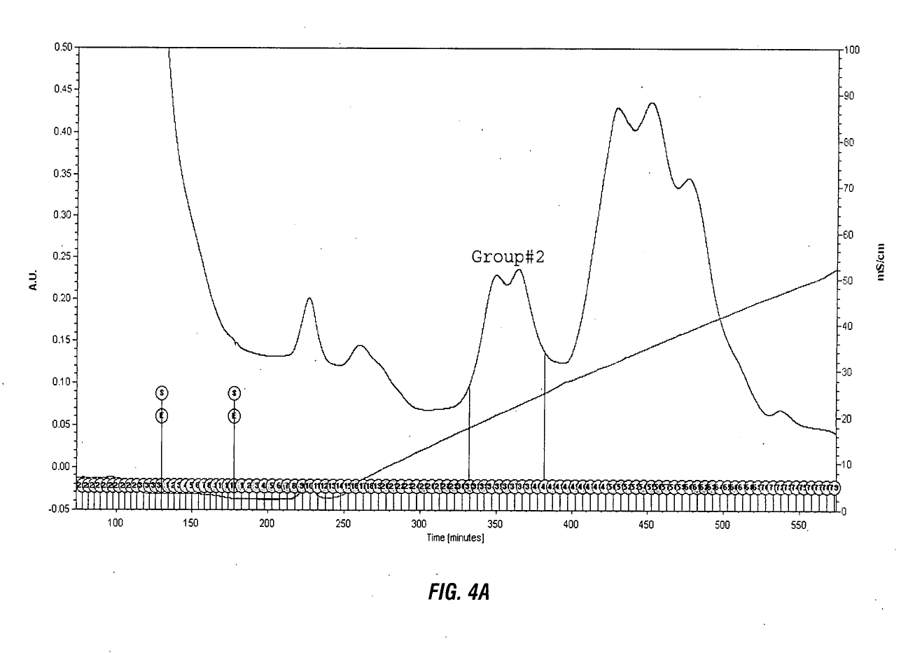

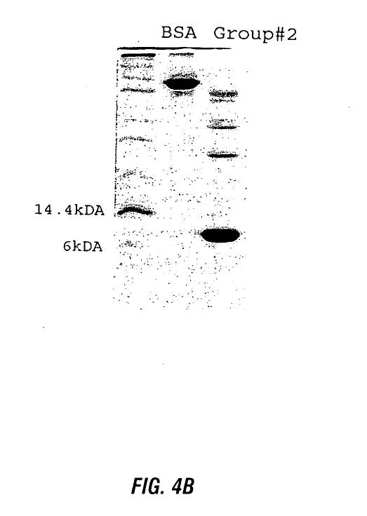

Native GR2s were extracted from maize pollen and purified by chromatography, as described in FIG. 4. The GR2 band contains several GR2 isoforms named Zea m2 or Zea m3, according to the standard allergen nomenclature. The synergistic effects noted here were also found for HPLC-purified Zea m2 and Zea m3, which were >90% pure. In additional experiments (not shown), pollen from rye grass (Lolium perenne) was used instead of maize pollen, to purify a GR2 known as Lol p3.

Recombinant Zea m2 (abbreviated ZM2) and Zea m3 (abbreviated ZM3) proteins were produced using standard methods in E. coli by use of pET22b(+) expression vector constructed with cDNAs for Zea m2 and Zea m3. The endogenous signal peptide was removed in these constructs and replaced with a methionine. Sequence 1 shows the nucleotide and protein sequences for recombinant Zea m2 and Sequence 2 shows the nucleotide and protein sequences for recombinant Zea m3. The recombinant proteins were column-purified as in FIG. 4.

Methods: Ten grams of frozen maize pollen was extracted in 40 mL of 50 mM sodium acetate, pH 4.5, for 1 hr at 4° C. The extract was centrifuged at 14,000 g at 4° C. and then loaded unto a CM-Sepharose column (15×300 m) equilibrated in 20 mM sodium acetate, pH 4.5. The column was washed with the same buffer until absorbance at 280 nm returned to baseline. Protein was eluted at 1 mL/min. with a linear gradient of NaCl (0-500 mM in 6 h) Fractions were collected at 5 mL per tube. For SDS-PAGE, samples were de-salted before loading onto the gel. Large band at ˜10 kD=GR2, indicated with arrow.

Cellulase Enzymes Used in This Work Are as Follows:

a. Trichoderma cellulase is a complex enzyme mixture purchased from Sigma (C-8546)

b. E5 is endoglucanase 5 from Thermomonospora fusca

c. E5cd is the catalytic domain of endoglucanase 5 from Thermomonospora fusca

d. EGVcore is the catalytic core of endoglucanase V from Humicola insolens

e. EG1 is endoglucanase I (Cel7A) from Trichoderma reesei

f. CBH1 is purified cellobiohydrolase I (Cel7B) from Trichoderma reesei.

g. E1 is endoglucanase 1 from Acidothermus cellulolyticus

Cellulose hydrolysis was measured as release of reducing sugars by the PABAH method (Lever, 1972) or total soluble sugars by the anthrone method (Mokrascb, 1954).

B. Experimental Results

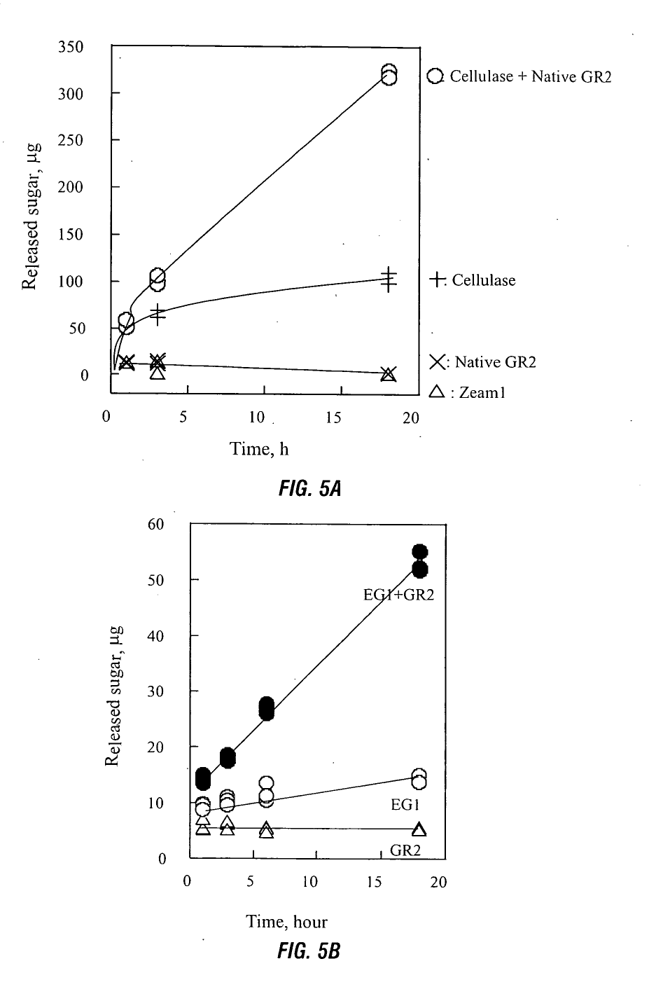

FIG. 5 (left panel) shows a time course for cellulose digestion by Trichoderma cellulase, with and without addition of native maize GR2 protein. Trichoderma cellulase is a potent mixture of enzymes with relatively strong ability to digest cellulose. The results show that native maize GR2 enhanced the cellulytic activity of Trichodermna cellulase, both in terms of the initial rate of enzyme digestion and digestion over longer times. A similar result was found when purified Acidothermus cellulolyticus E1 endoglucanase was used (FIG. 5, right panel)

It is generally accepted that the key rate-limiting step for cellulose hydrolysis by cellulases is the time required for solvation of a glucan on the surface of the cellulose microfibril; that is, to make the surface glucan accessible to enzymatic attack, it must be stripped from its ordered arrangement in the crystal. Thus, one interpretation of the results in FIG. 2 is that GR2 increases the accessibility of cellulose to the enzyme.

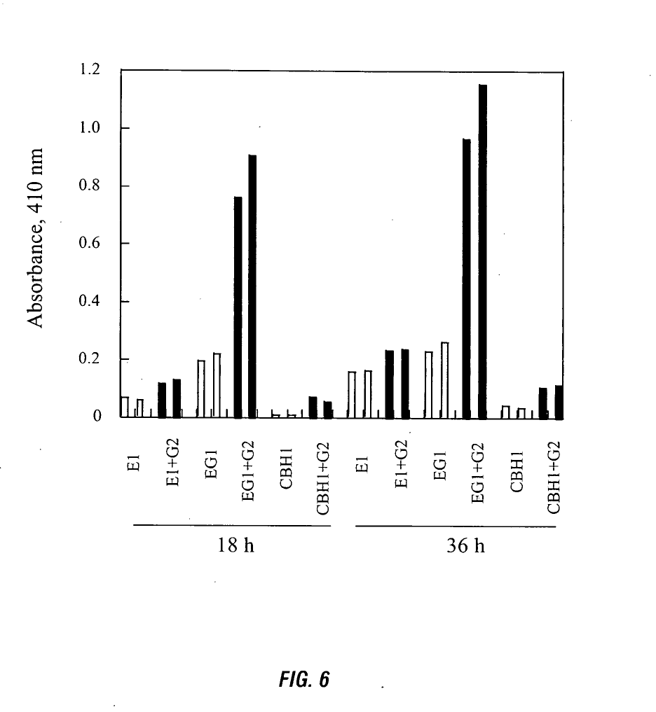

An alternative interpretation is that GR2 activates the enzyme itself. This is unlikely because it shows synergism with very divergent enzymes. We tested GR2 with a different single enzymes from other microbial sources. The results (Table 1; FIG. 6) demonstrate that GR2 has its synergistic effect with all the enzymes tested. Because these enzymes are diverse, are not homologous to one another, and come from different enzyme families, it is unlikely that GR2 activates the enzyme itself. A more reasonable interpretation is that GR2 acts on the cellulose substrate to make it more accessible to enzyme attack.

TABLE 1
Enhancement of cellulase activity by a maize GR2 (recombinant ZM3)
using various enzymes and comparison of its synergism with that of CBD
from Clostridium cellovorans (Sigma C-8581). 5 mg Avicel or filter
paper; 1 microgram cellulase; 10 micrograms ZM3 or 16 micrograms CBD

| | Released sugars (μg) | |
|---|---|---|
| Cellulases | Avicel | Whatman filter paper |
| E5 | 10.9 ± 1.3 | 9.71 ± 1.1 |
| E5 + ZM3 | 37.7 ± 4.6 (3.5) | 44.6 ± 3.4 (4.6) |
| E5 + CBD | 21.2 ± 0.7 (1.9) | 21.3 ± 3.1 (2.2) |
| E5 cd | 4.9 ± 1.8 | 7.2 ± 1.9 |
| E5 cd + ZM3 | 32.0 ± 6.1 (6.6) | 137.6 ± 1.0 (19.2) |
| E5 cd + CBD | 12.2 ± 1.6 (2.5) | 11.2 ± 1.6 (1.6) |
| EGV core | 2.2 ± 0.5 | 1.3 ± 0.2 |
| EGV core + ZM3 | 12.1 ± 1.6 (5.6) | 77.8 ± 2.4 (59.7) |
| EGV core + CBD | 10.6 ± 0.6 (4.9) | 19.4 ± 1.1 (14.9) |
| EGV intact | 3.9 ± 0.6 | 16.0 ± 2.7 |
| EGV intact + ZM3 | 18.3 ± 1.1 (4.7) | 57.1 ± 9.8 (3.6) |
| EGV intact + CBD | 10.8 ± 0.5 (2.8) | 48.0 ± 6.5 (3.0) |
| EG1 | 8.3 ± 0.4 | 10.8 ± 1.0 |
| EG1 + ZM3 | 49.7 ± 2.9 (6.0) | 131.1 ± 9.8 (12.1) |
| EG1 + CBD | 36.1 ± 2.0 (4.3) | 33.5 ± 3.3 (3.1) |
| CBHI | 4.4 ± 0.3 | 2.0 ± 0.6 |
| CBHI + ZM3 | 15.3 ± 0.3 (3.5) | 5.0 ± 1.2 (2.5) |
| CBHI + CBD | 11.1 ± 1.3 (2.5) | 5.0 ± 1.0 (2.5) |

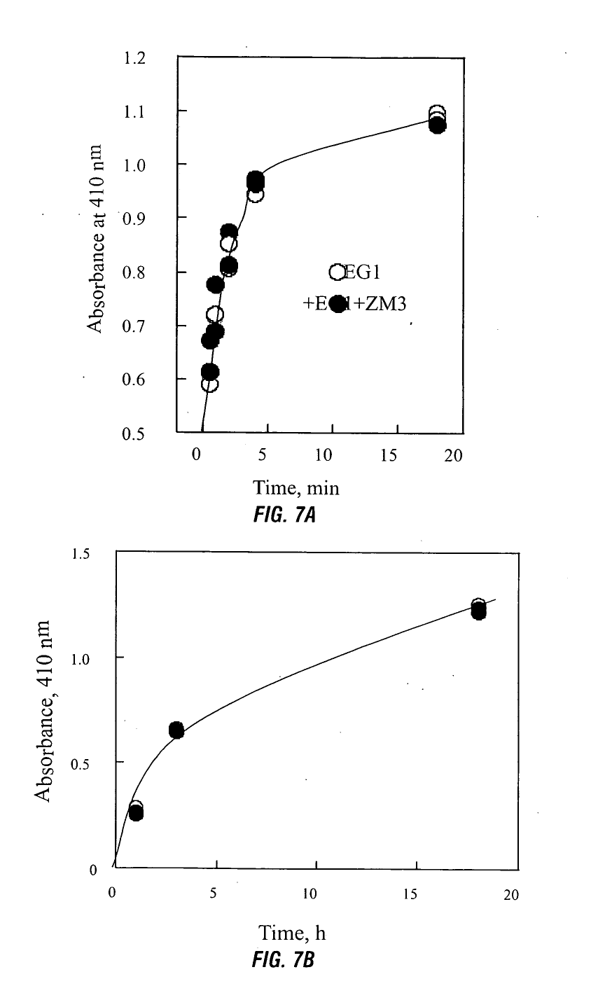

This interpretation is further supported by the results of hydrolysis assays using soluble glucans, where GR2 did not enhance the rate of hydrolysis of hydroxymethyl cellulose, a soluble cellulose derivative (FIG. 7A). Similar results were found with use of soluble xyloglucan, which has a glucan backbone as in cellulose, but with xylose branches which increase the solubility of the glucan (FIG. 7B). This result shows that GR2 does not activate cellulase enzyme activity per se, leaving us with the interpretation that GR2 acts on cellulose to enhance its hydrolysis by enzymes.

One potential variant explanation for the synergism by GR2 is that it frees cellulases that have become nonproductively-trapped in the cellulose substrate. Enzyme trapping has been offered as a potential explanation for the slowing down of the rate of hydrolysis with time. The data in FIG. 7 and Table 1, however, discount the hypothesis that GR2 causes its synergistic effect by freeing entrapped enzyme. The reasoning is as follows: Cellulases with cellulose-binding domains (CBD) show a greater propensity for entrapment than enzymes without such domains. Thus, if GR2 synergism is mainly due to the freeing of trapped enzyme, the synergistic effect of GR2 should be greater for cellulases with CBDs. E5 has a CBD, whereas E5cd lacks the CBD. As shown in table 1, GR2 gives greater synergism with CBD than with E5. A similar conclusion is drawn from the comparison of GR2's effect with EGVcore (which lacks a CBD) and EGVintact, which has its CBD.

Table 1 also shows the synergism caused by CBD from Clostridium cellovorans (Sigma C-8581). Two points can be made about these data. First, this bacteria CBD also demonstrates synergistic effect. This is a new result. Second, ZM3 has a superior synergism, in some cases markedly so (compare the results with Whatman filter paper).

To account for the slow down of the cellulose hydrolysis reaction with time (see FIG. 5), it has been proposed that cellulases at first more readily hydrolyze the less crystalline regions of the cellulose microfibril, leaving the more crystalline regions that are more resistant to enzymatic attack. We see from the time course in FIG. 5 that the most striking effect of GR2 is obtained with prolonged digestion, at the later times when the cellulose forms more recalcitrant to hydrolytic attack are more prevalent.

From the above results we conclude that GR2 makes cellulose more accessible to enzymatic attack and thereby synergistically enhances cellulase action.

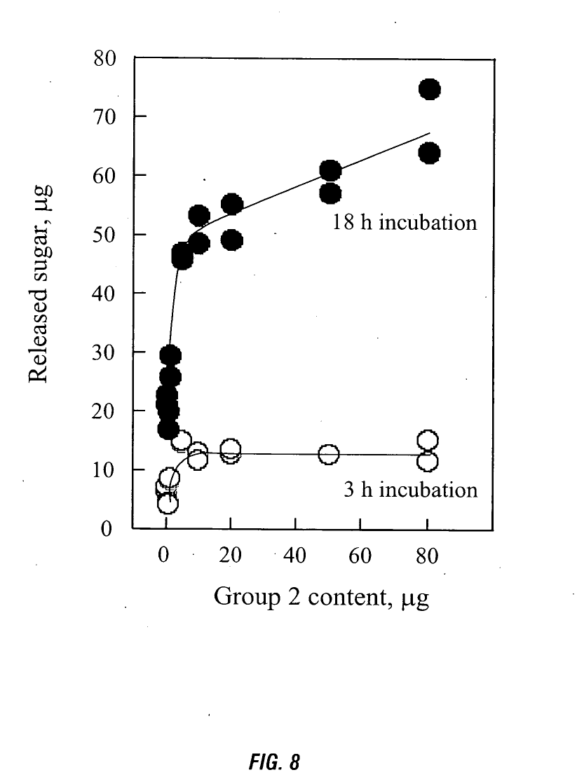

To determine the amount of GR2 needed for this synergistic effect, we measured hydrolysis by Trichioderma cecellulase as a function of increasing GR2 contents, using two time points (3 h and 18 h, see FIG. 8). There is a steep increase in hydrolysis in the range of 0-5 μg of GR2; thereafter there is a slower increase with increasing GR2 levels. Note that at 10 μg GR2 (which is 1/10^{th} the amount of cellulase that is used in this assay), the reaction rate is increased 3-4 fold at 18 h. This result indicates that relatively small amounts of GR2 can substantially increase cellulase action.

We tested a variety of cellulose substrates, with varying degrees of crystallinity (Table 2). The synergistic effect of GR2 was observed with all of the substrates, with cellulose of high crystallinity (cotton, Whatman filter paper, Avicel) showing a greater % enhancement as compared with cellulose of low crystallinity (phosphoric acid swollen celluloses from these sources). This result is consistent with the conclusion that GR2 stimulates cellulase action by increasing glucan accessibility. The phospho-swollen celluloses are more accessible than the crystalline celluloses, and therefore the GR2 effect is somewhat reduced in them. However, note that there is still a 2-3 fold enhancement by GR2 even in these swollen forms of cellulose.

TABLE 2
GR2 synergism with various cellulose substrates.

| | Released sugar (μg) | | Ratio |
|---|---|---|---|
| Substrate | EG1 | EG1 + ZM3 | (EG1 + ZM3)/EG1 |
| Cotton linter | 3.1 ± 0.7 | 29.0 ± 2.4 | 9.3 |
| Cotton phospho-swollen | 195.6 ± 43.1 | 439.9 ± 50.7 | 2.2 |
| Cotton alkaline-swollen | 7.6 ± 0.1 | 36.3 ± 1.7 | 4.8 |
| Avicel | 7.6 ± 1.0 | 50.4 ± 5.9 | 6.6 |
| Avicel phospho-swollen | 85.8 ± 14.1 | 247.3 ± 21.3 | 2.9 |
| Whatman filter paper | 9.9 ± 1.2 | 98.6 ± 14.4 | 10.0 |
| Whatmanfiter paper phospho-swollen | 93.3 ± 23.5 | 336.3 ± 29.5 | 3.6 |

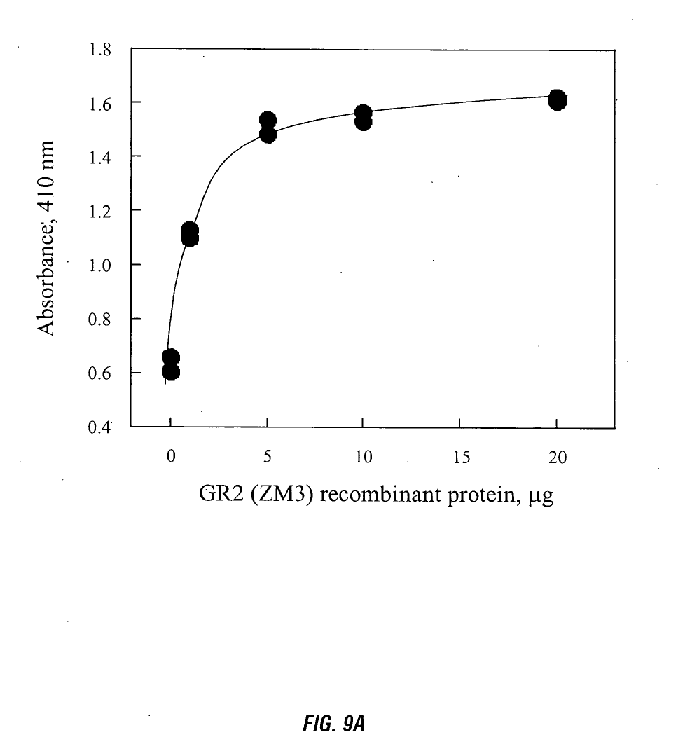

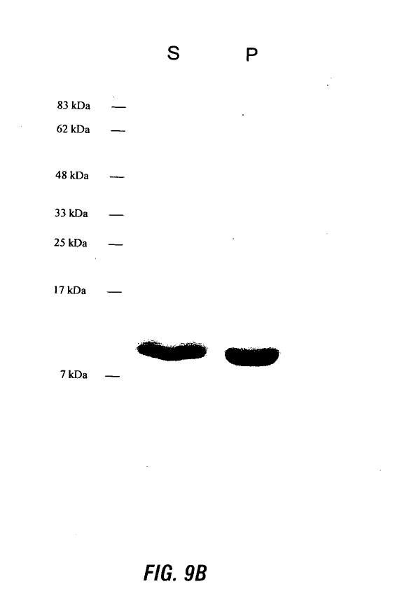

To verify that the synergistic effect is indeed due to GR2 protein, we expressed recombinant Zea m2 and Zea m3 (two different forms of maize GR2 proteins) in E. coli by use of the pET expression vector and tested the recombinant protein for synergism. As shown in FIG. 9, the recombinant Zea m3 exhibited strong synergistic effect. Likewise recombinant Zea m2 had synergistic activity. Moreover, native Lol p3, purified from ryegrass pollen showed similar synergistic activity. As pointed out above, sequences of these proteins are very divergent (as low as 22% identity).

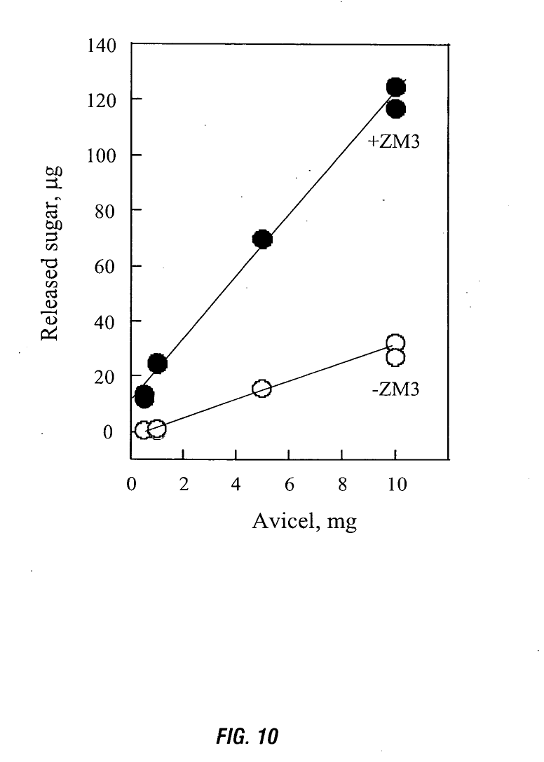

The synergistic activity of GR2 was found over a wide range of substrate concentrations (FIG. 10).

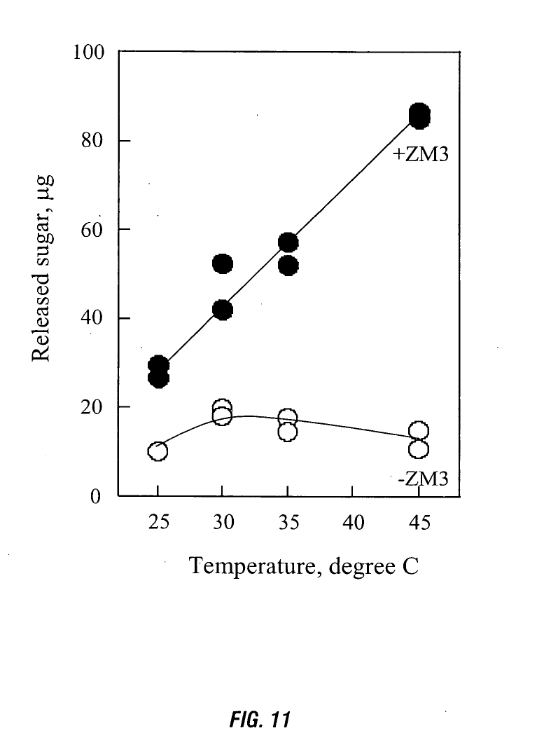

We tested the synergistic activity of GR2 over a range of temperatures, and found that within the temperature range tested, the synergistic effect was greater at the higher temperatures (FIG. 11). Note that at 45 C, the synergistic activity was 3-4 fold higher than at 25 C.

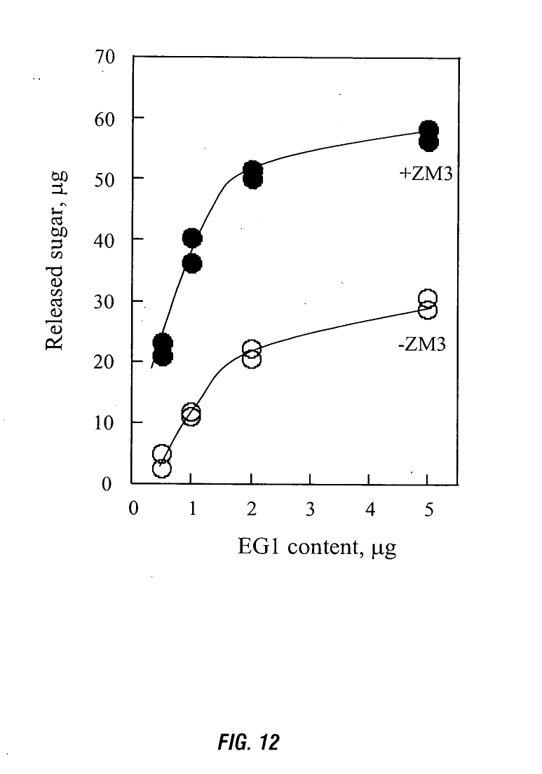

When tested at varying enzyme concentrations, the synergistic effect of GR2 was observed at all concentrations, but the relative effect (the ratio) was higher at lower enzyme concentrations (FIG. 12).

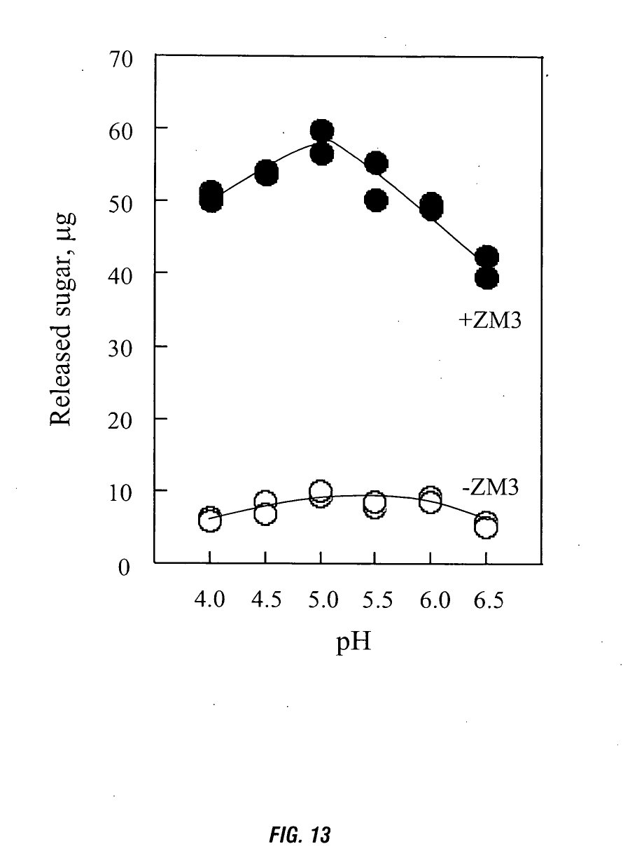

When tested at varying pH, the synergistic effect of GR2 was found throughout the pH range of 4 to 6.5, with an apparent (broad) optimum at pH 5 (FIG. 13).

The synergistic action of GR2 was also observed in measures of alkali-soluble sugars bound to cellulose after cellulase digestion (Table 3). Alkali-soluble sugar are presumably oligosaccharides with degree of polymerization >5. The results indicate that GR2 increases the number of cuts made in a cellulose backbone by cellulase.

TABLE 3
Synergistic action of recombinant ZM3 on alkali-soluble sugars bound
to cellulose.

| | Buffer soluble | Alkaline soluble |
|---|---|---|
| | (μg) | |
| Cotton linter + EG1 | 41.8 ± 6.4 | 94.83 ± 15.6 |
| Cotton linter + EG1 + ZM3 | 160.1 ± 22.6 | 153.3 ± 45.1 |
| Whatman paper + EG1 | 44.9 ± 5.2 | 153.8 ± 4.6 |
| Whatman paper + EG1 + ZM3 | 146.7 ± 2.4 | 358.5 ± 20.5 |

Methods: Cellulose (Cotton and Whatman paper) pre-treated with 24% KOH plus 0.1% NaBH4 followed by neutralized with acetic acid was incubated with EG1 (1 μg) and ZM3 (5 μg) for 18 hours. The supernatant was used for measuring released sugars. The insoluble cellulose was washed with sodium acetate buffer (50 mM, pH 4.5) and treated with 24% KOH plus 0.1% NaBH4 for 1 hour, and then the supernatant was neutralized with acetic acid and measured sugar content by the anthrone method.

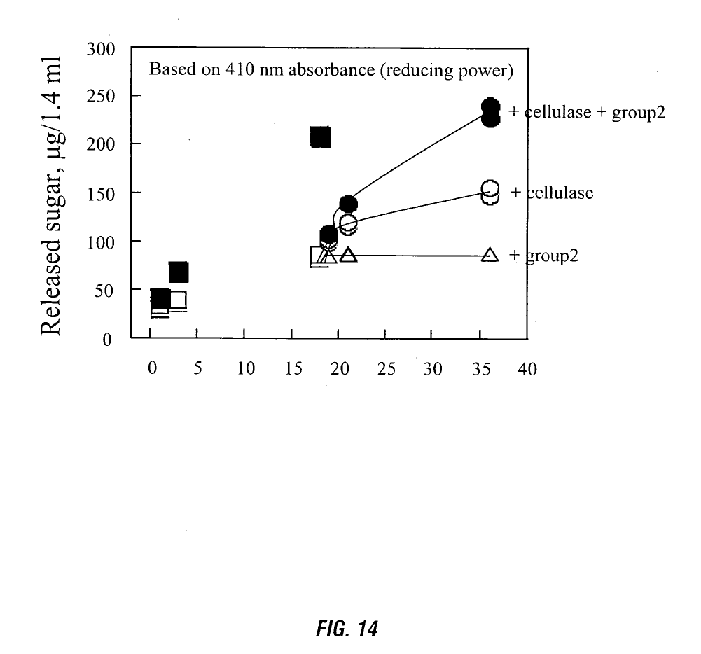

It is possible that GR2 could cause release of such oligosaccharides from the microfibril. This idea was tested by an experiment in which cellulase digestion was allowed to proceed for 18 h, and then the buffer was replaced with one containing only GR2, or cellulase, or both (FIG. 14). The results show that when GR2 alone was swapped in, little or no additional sugars were released. Thus we do not find evidence that GR2 alone can cause release of longer oligosaccharides from the cellulose microfibrils.

The data in Table 4 demonstrate that ZM3 is a very robust protein and activity survives very harsh treatments, including autoclaving and hot urea treatments. GR2 synergistic activity was also found in protein purified by reverse-phase chromatography using acetonitrile or methanol as the organic eluant. Thus, GR2 activity can survive exposure to organic solvents as well.

TABLE 4
Does the synergistic action of ZM3 survive denaturing conditions.

| Treatment | Released sugar, μg |
|---|---|
| EG1 | 12.9 ± 1.7 |
| EG1 + ZM3 | 53.3 ± 1.4 |
| EG1 + heated ZM3 | 51.8 ± 3.3 |
| EG1 + autoclaved ZM3 | 47.4 ± 1.2 |
| EG1 + hot urea treated ZM3 | 48.7 ± 2.6 |
| EG1 + protease treated ZM3 | 14.7 ± 2.0 |

Methods: Avicel (5 mg) was incubated with EG1 and ZM3, which was heated, autoclaved, Hot urea treated or protease (Streptomyces, 100 μg) treated, for 18 h at 30° C., and the supernatant was measured total sugar by anthrone/H₂SO₄ method. Urea was removed with Centricon 5 kD and protease was inactivated by heat at 95° C. for 15 min.

The results in Table 5 indicate that at least some ZM3 binds tightly to cellulose, since washing with buffer or NaCL solution did not eliminate the synergistic effect.

TABLE 5
Synergism by ZM3 persists after washing and is not greatly
inhibited by NaCl.

| Treatment | Released sugar, μg |
|---|---|
| EG1 | 12.4 ± 1.4 |
| EG1 + ZM3 | 52.1 ± 0.9 |
| EG1 + ZM3 (5 min incubation) | 27.9 ± 2.4 |
| EG1 + ZM3 (30 min incubation) | 34.5 ± 1.2 |
| EG1 + ZM3 (120 min incubation) | 47.7 ± 1.5 |
| EG1 + ZM3 + 100 mM NaCl | 41.4 ± 1.2 |
| EG1 + ZM3 + 1M NaCl | 40.0 ± 1.0 |

Methods: Walls were incubated with ZM3 for the times indicated, then washed with fresh buffer and EG1 added. Where indicated, NaCl was added to test for salt inhibition.

Synergism between EGI and CBHI has been noted in the published literature (Henrissat et al., 1985). Table 6 confirms this synergism and demonstrates the comparatively strong synergism by ZM3.

TABLE 6
Synergism of GR2 with CBHI and EG1.

Released sugar, μg

| Treatment | Avicel | Whatman paper |
|---|---|---|
| EG1 | 8.0 ± 0.7 | 24.5 ± 1.4 |
| CBHI | 4.3 ± 0.2 | 1.38 ± 0.2 |
| EG1 + CBHI | 17.2 ± 1.2 | 32.1 ± 2.5 |
| EG1 + ZM3 | 50.7 ± 1.1 | 165.5 ± 5.2 |
| CBHI + ZM3 | 14.7 ± 0.8 | 8.4 ± 1.3 |
| EG1 + CBHI + ZM3 | 73.5 ± 3.9 | 265.3 ± 14.1 |

Avicel and Whatman filter paper (5 mg) was incubated with EG1, CBHI and ZM3 at 30° C. for 18 hours, and released sugar was measured by anthrone/H₂SO₄ Method. One μg of enzyme and 5 μg of ZM3 were used for this assay.

GR2 synergism not only works with pure cellulose, but also more complex cell walls, such as those from young seedlings (Table 7).

TABLE 7
GR2 synergism in whole cell walls from seedlings.
Digestion of maize and cucumber cell wall by cellulase

| | Released sugars, μg | Absorbance at 640 nm |
|---|---|---|
| 1) Maize cell wall + EG1 | 0.5098, 0.4969 | 156.40, 152.45 |
| 2) Maize cell wall + EG1 + ZM3 | 0.7586, 0.8693 | 232.74, 266.70 |
| 3) Cucumber cell wall + EG1 | 0.1306, 0.1287 | 40.07, 39.48 |
| 4) Cucumber cell wall + EG1 + ZM3 | 0.3487, 0.3259 | 106.98, 99.98 |

Maize (2 mg) and cucumber (2 mg) cell walls (Phenol-Acetic acid-Water washed) were pretreated with EG1 (1 μg) for 18 hours at 30° C., heated, washed with sodium acetate buffer (50 mM, pH 4.5) and incubated with EG1 (1 μg) and ZM3 (5 μg) at 30° C. for 18 hours.

In Table 8 we find evidence that ZM3 does not cause a major, irreversible change in cellulose structure (such as cracking open large new surfaces for action by cellulases). This is evidenced from the fact that ZM3 pretreatment of cellulose, followed by protease treatment, eliminates the synergistic effect.

TABLE 8
Effect of Protease treatment of ZM3-treated cellulose.

| | Released sugars, μg |
|---|---|
| Absorbance at 410 nm // at 640 nm | (calculated from the absorbance at 640 nm) |
| 1) 0.0401, 0.0375 // 0.0326, 0.0343 | 15.00, 15.78 |
| 2) 0.2026, 0.2188 // 0.1339, 0.1480 | 61.62, 68.10 |
| 3) 0.0455, 0.0468 // 0.0332, 0.0364 | 15.28, 16.75 |
| 4) 0.0390, 0.0364 // 0.0365, 0.0388 | 16.79, 17.86 |
| 5) 0.2178, 0.2219 // 0.1446, 0.1329 | 66.54, 61.16 |

Avicel (5 mg) in sodium acetate buffer (500 μl, 50 mM, pH 4.5) was incubated with recombinant ZM3 protein (5 μg) for 1 hour, treated with Protease (100 μg) at 37° C. for 1 hour, heated at 95° C. for 15 min, and incubated with EG1 (1 μg) at 30° C. for 18 hours.
1) Avicel ± EG1
2) Avicel + EG1 + ZM3
3) Avicel pretreated with ZM3 followed by treatment with protease as described above + EG1
4) Avicel + ZM3 pretreated with protease + EG1
5) Avicel + ZM3 + EG1 + inactivated protease solution

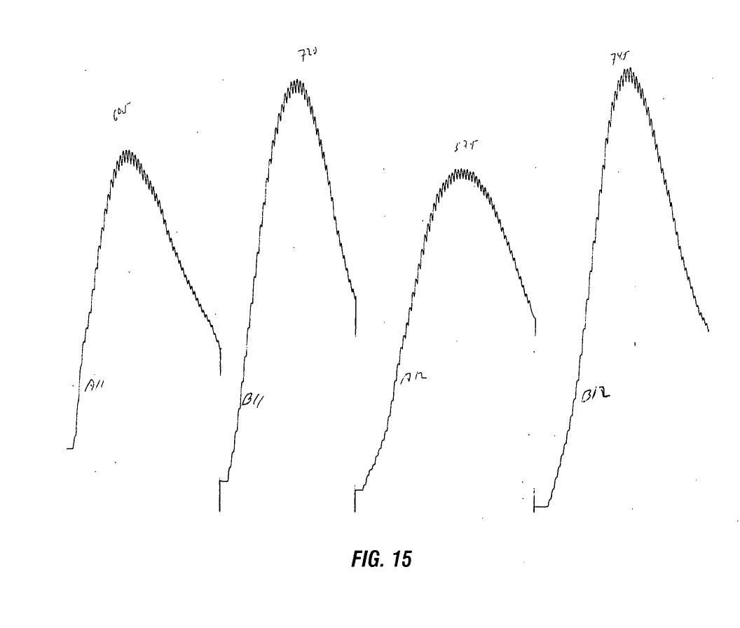

Pretreatment of Whatman filter paper strips with maize GR2 results in substantial weakening of the paper, as measured in force-extension curves (FIG. 15).

Native maize GR2 is able to cause release of Direct Cotton dyes bound to cellulose (Table 9). The largest effects in this assay were found with Direct Red 2, Direct Red 80, and Congo Red. Small or negligible effects were found with Pontamine fst Orange 6RN, which is a different class of dye. Because Congo Red and related dyes are believed to bind to the surface of cellulose via hydrogen bonding, these result indicate that GR2 proteins are able to hydrogen-bonded substances from the surfaces of polysaccharides such as cellulose.

TABLE 9
Release of Direct Cotton Dyes from paper by maize GR2

Protocol:
1. Pre-stain punched disks of Whatman #1 with .1% dyes* for 1 h, wash
well
2. Put 5 stained disks in 2-mL eppendorf tube
3. Add 0.7 mL 20 mM Na acetate pH 5.5 +/− 70 ug maize GR2, stir at
900 RPM at 30 C
4. Spin high speed 1 min, take 600 uL and measure in spectrophotometer
at XX nm; replace solution in tube and continue agitation
Dyes:
1. Congo Red at 1 mg/mL; Amax = 485 nm
2. Direct Blue 1 at 1 mg/mL; Amax = 638 nm
3. Pontamine fast Orange 6RN at 5 mg/mL; Amax = 430 nm
4. Direct Red 80 at 1 mg/mL; Amax = 525 nm
5. Direct Red 2 at 1 mg/mL′ Amax = 490 nm

Direct release, measure in Absorbance, for 5 dyes +/− maize
GR2 at three time points (20 min, 90 min and 3 h).

RESULTS

| | | 1(486) | 2(638) | 3(430) | 4(525) | 5(486) |
|---|---|---|---|---|---|---|
| 20 MIN | + | 0.4149 | 0.192 | 0.1348 | 0.171 | 0.1299 |
| | − | 0.1233 | 0.1078 | 0.098 | 0.075 | 0.0128 |
| 90 MIN | + | 0.513 | 0.1969 | 0.1 | 0.1686 | 0.1533 |
| | − | 0.1229 | 0.142 | 0.1129 | 0.0974 | 0.0142 |
| 3 H | + | 0.454 | 0.1815 | LOST | 0.2082 | 0.262 |
| | − | 0.1157 | 0.0928 | 0.1463 | 0.0672 | 0.024 |

### REFERENCES

Ansari A A, Freidhoff L R, Marsh D G. 1989a. Molecular genetics of human immune responsiveness to Lolium perenne (rye) allergen, Lol p III [published erratum appears in Int. Arch Allergy Appl Immmnol 1989 Sep;50(9):following 424]. International Archives of Allergy & Applied Immunology 88: 164-169.
Ansari A A, Ponniah S, Marsh D G. 1989b. Complete amino acid sequence of a Lolium perenne (perennial rye grass) pollen allergen, Lol p II. Journal of Biological Chemistry 264: 11181-11185.
Ansari A A, Shenbagamurthi P, Marsh D G. 1989c. Complete primary structure of a Lolium perenne (perennial rye grass) pollen allergen, Lol p III: comparison with known Lol p I and II sequences. Biochemistry 28: 8665-8670.
Brown R M, Jr., Saxena I M, Kudlicka K. 1996. Cellulose biosynthesis in higher plants. Trends in Plant Science 1: 149-155.
De Marino S, Morelli M A, Fraternali F, Tamborini E, Musco G, Vrtala S, Dolecek C, Arosio P, Valenta R, Pastore A. 1999. An immunoglobulin-like fold in a major plant allergen: the solution structure of Phl p 2 from timothy grass pollen. Structure Fold Des 7: 943-952.
Dolecek C, Vrtala S, Laffer S, Steinberger P, Kraft D, Scheiner 0, Valenta R. 1993. Molecular characterization of Phl p II, a major timothy grass (Phleum pratense) pollen allergen. FEBS Letters 335: 299-304.
Durbin R, S. Eddy S, Krogh A, Mitchison G. 1998. Biological Sequence Analysis: Probabilistic Models of Proteins and Nucleic Acids, Cambridge University Press,
Fedorov A A, Ball T, Valenta R, Almo S C. 1997. X-ray crystal structures of birch pollen profilin and Phl p 2. International Archives of Allergy and Immunology 113: 109-113.
Henrissat B, Driguez H, Viet C, Schulein M. 1985. Synergism of cellulases from Trichoderma reesei in the degradation of cellulose. Bio-Technology 3: 722-726.
Hon D N S, Shiraishi N. 2000. Wood and Cellulosic Chemistry, Second Edition, Revised, and Expanded. MARCEL DEKKER—TAYLOR & FRANCIS—CRC.
Kende H, Bradford K, Brummell D, Cho H T, Cosgrove D J, Fleming A, Gehring C, Lee Y, Queen-Mason S, Rose J, Voesenek L A. 2004. Nomenclature for members of the expansin superfamily of genes and proteins. Plant Molecular Biology 55: 311-314.
Lever M. 1972. A new reaction for colorimetric determination of carbohydrates. Analytical Biochemistry 47: 273-279.
Mokrasch L C. 1954. Analysis of Hexose Phosphates and Sugar Mixtures with the Anthrone Reagent. Journal of Biological Chemistry 208: 55-59.

Sampedro J, Cosgrove D J. 2005. The expansin superfamily. Genome Biol. 6: 242.

Sequence 1:
nucleotide and protein sequence for ZM2
cDNA (underlined sequence = coding region)
TCCATCCATCCATCCATCCATCCCTAAAAATCAAGGCTACACACCAACTT
ACTTTCTAGGTCTCAAATTAA
ATGGCCTCCTCGTCCTCCTCCTTGCTGCTGGCGTCGATGGCGGTGGCGGC
ACTCTTTGTCGTCGGCTCGTGTGGCGGCGCGCTCACCTTCACGATCGGCA
AGGACTCCAGCTCCACCAAACTATCCCTCGTCACTAACGTTGCCATCTCC
GAGGTGTCGGTCAAGGAGAAGGGCGCCCTGGACTGGTCGGATGATCTCAA
GGAGTCGCCAGCCAAAACCTTCACCCTCGATAGCAAGGAGCCGATTAAGG
GCCCCATCTCCGTCCGCTTCGCTGTGAAAGGTGGTGGCTACCGCGTCGTT
GATGACGTCATCCCTGCCGACTTCAAGCCTGGCTCTGTTTACCAGACAGG
CGAACAAATC
TGAGTAATGGATTCTGCTGCGTGCAGATTATATTGATCTCTAAAATAAAT
GTTTGACAGAGACTAATTAAGCACACAAACAAAGCAAGACACTAATTCAT
TTCCTATGTTTACTTTGTGGTAGGCAACTGCAAAACAATGTATTGTAT
Protein (underlined sequence = signal peptide;
remained = mature protein of 94 residues, 10.0 kD;
Isolectric Point = 5.2)
MASSSSSLLLASMAVAALFVVGSCGG
ALTFTIGKDSSSTKLSLVTNVAISEVSVKEKGALDWSDDLKESPAKTFTL
DSKEPIKGPISVREAVKGGGYRVVDDVIPADFKPGSVYQTGEQI
Sequence 2:
nucleotide and protein sequence for ZM3
cDNA
GCACGAGACACACATCCGTGAAAGAAACAAGCACATAGAAACAAATACAT
CGCTAGCTCGCCACCAACA
ATGGCCTCAAGGTACTCTATCCTGCTTGCCACAACGGCACTGGCTATGTT
GTTCGCATTCGGTTCGTGCACCACCCCACTCACCTTCCAGGTCGGCAAGG
GCTCCAAGCCTGGCCACCTGGTTCTCACCCCTAACATTGCCACCATCTCT
GACGTGGAGATCAAGGAGCATGGCGGCGACGATTTCTCCTTTACACTCAA
GGAGGGCCCAGCTGGCACTTGGACGCTCGACACCAAGGCCCCGCTCAAGT
ACCCCCTCTGCATCCGCTTTGCTACCAAGTCTGGCGGCTACCGTATCGCC
GATGATGTCATCCCCGCCGATTTCAAGGCTGGCACCACCTACAAGACCAC
TCTCAGCATC
TAATCAGCCTCTGATAAGTGATGACGAATAATATTTCGAAAGAGCTCATC
TAGTCCACGTGCCAACGAGCCAATATTTAAATTTTTTCTATGGTTATTTT
TGTGGCACAACACCATCTCTTTCTGTGCCTTGTTGCGTTGGCTGACTTTA
TTACATGAATTGAAATACACTGCTTATTAAGA
Protein (underlined sequence = signal peptide;
remained = mature protein of 97 residues, 10.4 kD;
Isolectric Point = 8.1)
MASRYSILLATTALAMLFAFGSC
TTPLTFQVGKGSKPGHLVLTPNIATISDVEIKEHGGDDFSFTLKEGPAGT
WTLDTKAPLKYPLCIRFATKSGGYRIADDVIPADFKAGTTYKTTLSI

What is claimed is:

1. A method of enhancing the hydrolysis of cellulose by cellulase comprising: introducing to said cellulose a GR2 protein in the presence of said cellulase.

2. The method of claim 1 wherein said hydrolysis of cellulose is in the conversion to ethanol or other biofuel.

3. The method of claim 1 wherein said cellulase is one which has been isolated from bacteria.

4. The method of claim 1 wherein said cellulase is one which has been isolated from fungi.

5. The method of claim 1 wherein said cellulase is one which is isolated from Trichoderma.spp, Aspergillus spp, or Humicol spp.

6. The method of claim 1 wherein said cellulase is selected from the group consisting of: Trichoderma cellulase, endoglucanase 5 from Thermomonospora fusca; the catalytic domain of endoglucanase 5 from Thermomonospora fusca; the catalytic core of endoglucanase V from Humicola insolens; endoglucanase I (Cel7A) from Trichoderma reesei; cellobiohydrolase I (Cel7B) from Trichoderma reesei; and endoglucanase 1 from Acidothermus cellulolyticus.

7. The method of claim 1 wherein said cellulose is present in wood.

8. The method of claim 1 wherein said cellulose is present in paper.

9. The method of claim 1 wherein said cellulose is present in a plant cell wall.

10. The method of claim 1 wherein said GR2 protein is one from pfam01357.

11. The method of claim 1 wherein said GR2 protein is a group 2 grass pollen allergen protein.

12. The method of claim 1 wherein said GR2 protan is a group 3 grass pollen allergen.

13. The method of claim 1 wherein said GR2 protein is recombinant.

14. The method of claim 1 wherein said GR2 protein contains the domain 2 of an expansin protein and is not an expansin.

15. The method of claim 1 wherein said (NCBI Protein Accession #PI 4947), Lol p3, Ph1 p2, Zea m2, and Zea m3.

16. A method of altering the mechanical strength of cellulosic materials comprising:
introducing to cellulosic materials a cellulase and a GR2 protein.

17. The method of claim 16 wherein said cellulosic materials are one or more of the following: paper, cloth, composites, or wood.

18. The method of claim 16 wherein said cellulase is one which has been isolated from bacteria.

19. The method of claim 16 wherein said cellulase is one which has been isolated from fungi.

20. The method of claim 16 wherein said cellulase is one which is isolated from Trichoderma.spp, Aspergillus spp, or Humicol spp.

21. The method of claim 16 wherein said cellulase is selected from the group consisting of: Trichoderma cellulase, endoglucanase 5 from Thermomonospora fusca; the catalytic domain of endoglucanase 5 from Thermomonospora fusca; the catalytic core of endoglucanase V from Humicola insolens; endoglucanase I (Cel7A) from Trichoderma reesei; cellobiohydrolase I (Cel7B) from Trichoderma reesei; and endoglucanase 1 from Acidothermus cellulolyticus.

22. The method of claim 16 wherein said GR2 protein is one from pfam01357.

23. The method of claim 16 wherein said GR2 protein is a group 2 grass pollen allergen protein.

24. The method of claim 16 wherein said GR2 protein is a group 3 grass pollen allergen.

25. The method of claim 16 wherein said GR2 protein is recombinant.

26. The method of claim 16 wherein said GR2 protein contains the domain 2 of expansin protein and is not an expansin.

27. The method of claim 16 wherein said (NCBI Protein Accession #P14947), Lol p3, Phl p2, Zea m2, and Zea m3.

28. The method of claim 16 wherein said GR2 is produced in plants.

29. A method of increasing the accessibility of cellulose for chemical modification comprising:
introducing to said cellulose an effective amount of a GR2 protein.

30. A method of improving the dye release from cellulosic materials (paper, cloth,) comprising:
introducing a GR2 protein to said materials which have dye hydrogen bonded to said cellulose.

31. A composition for improving the degradation of cellulose by cellulase comprising
a GR2 protein and a carrier.

32. The composition of claim 31 wherein said (NCBI Protein Accession #P14947), Lol p3, Phl p2, Zea m2, and Zea m3.

33. The composition of claim 31 wherein said GR2 protein is one from pfam01357.

34. The composition of claim 31 wherein said GR2 protein is a group 2 grass pollen allergen protein.

35. The composition of claim 31 wherein said GR2 protein is a group 3 grass pollen allergen.

36. The composition of claim 31 wherein said GR2 protein is recombinant.

37. The composition of claim 31 wherein said GR2 protein contains the domain 2 of expansin protein and is not an expansin.

38. The composition of claim 31 wherein said (NCBI Protein Accession #P14947), Lol p3, Phl p2, Zea m2, and Zea m3.

39. A cellulose degrading composition comprising:
a GR2 protein, and a cellulase.

40. The composition of claim 39 wherein said GR2 protein is one from pfam01357.

41. The composition of claim 39 wherein said GR2 protein is a group 2 grass pollen allergen protein.

42. The composition of claim 39 wherein said GR2 protein is a group 3 grass pollen allergen.

43. The composition of claim 39 wherein said GR2 protein is recombinant.

44. The composition of claim 39 wherein said GR2 protein contains the domain 2 of expansin protein and is not an expansin.

45. The composition of claim 39 wherein said (NCBI Protein Accession #P14947), Lol p3, Phl p2, Zea m2, and Zea m3.

46. A method for improving the enzymatic hydrolysis of glucan bonds in polysaccharides such as cellulose comprising:
introducing a GR2 protein to said enzyme in the presence of said polysaccharide.

47. The method of claim 46 wherein said GR2 protein comprises a domain 2 region from an expansin protein.

48. The method of claim 46 wherein said GR2 protein is one from pfam01357.

49. The method of claim 46 wherein said GR2 protein is a group 2 grass pollen allergen protein.

50. The method of claim 46 wherein said GR2 protein is a group 3 grass pollen allergen.

51. The method of claim 46 wherein said GR2 protein is recombinant.

52. The method of claim 46 wherein said GR2 protein contains the domain 2 of expansin protein and is not an expansin.

53. The method of claim 16 wherein a said cellulase and said GR2 protein are added in the form of a natural or recombinant chimeric protein containing both cellulase and GR2 domains.

54. The method of claim 31 wherein a said cellulase and said GR2 protein are added in the form of a natural or recombinant chimeric protein containing both cellulase and GR2 domains.

55. The method of claim 39 wherein a said cellulase and said GR2 protein are added in the form of a natural or recombinant chimeric protein containing both cellulase and GR2 domains.

56. The method of claim 46 wherein a said cellulase and said GR2 protein are added in the form of a natural or recombinant chimeric protein containing both cellulase and GR2 domains.

57. A novel chimeric protein for improving the degradation of cellulose by cellulase comprising:
a chimeric protein which contains both cellulase and GR2 domains.

58. The protein of claim 57 further comprising a lignase.
